(19) United States
(12) Patent Application Publication
(10) Pub. No.: US 2002/0012922 A1
(43) Pub. Date: Jan. 31, 2002

(54) Simplified method for indexing and determining the relative concentration of expressed messenger RNAs

(76) Inventors: Brian S. Hilbush, San Diego, CA (US); Karl W. Hasel, Solana Beach, CA (US); J. Gregor Sutcliffe, Cardiff, CA (US); Hwai Wen Chang, San Marcos, CA (US); Marie Lei A. Callahan, San Diego, CA (US); Jeanette Quan, San Diego, CA (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/775,217

(22) Filed: Feb. 1, 2001

Related U.S. Application Data
(63) Continuation-in-part of application No. 09/186,869, filed on Nov. 4, 1998.

(30) Foreign Application Priority Data
Oct. 14, 1999 (US) PCT/US99/23655

(51) Int. Cl. C12Q 1/68

(52) U.S. Cl. 435/6

(57) ABSTRACT

The invention provides a simplified method for simultaneous sequence-specific identification of multiple mRNA molecules in a mRNA population that eliminates the necessity of making a library in a vector. The invention also provides compositions and data processing systems suitable for the practice of the invention.

### CROSS REFERENCE TO RELATED APPLICATIONS

This application is a continuation-in-part of co-pending U.S. application Ser. No. 09/186,869, filed Nov. 04, 1998 and international application serial number PCT/US99/23655, filed Oct. 14, 1999, the teachings of which are incorporated by reference in their entirety.

### FIELD OF THE INVENTION

This invention relates generally to the field of gene expression and more specifically is directed to methods and compositions for the simultaneous identification and quantification of differentially expressed mRNA species.

### BACKGROUND OF THE INVENTION

A complete characterization of the protein molecules that make up an organism would be useful, e.g. for the improved design of drugs, the selection of optimal treatment of individual patients, and for the development of more compatible biomaterials. Such a characterization of expressed proteins would include their identification, sequence determination, demonstration of their anatomical sites of expression, elucidation of their biochemical activities, and understanding of how these activities determine organismic physiology. For medical applications, the description should also include information about how the concentration of each protein changes in response to pharmaceutical or toxic agents.

What is most needed to advance a chemical understanding of physiological function is a menu of protein sequences encoded by the genome plus the cell types in which each is expressed. At present, protein sequences can be reliably deduced only from cDNAs, not from genes, because of the presence of intervening sequences (introns) in the genomic sequences. Even the complete nucleotide sequence of a mammalian genome will not substitute for characterization of its expressed sequences. Therefore, a systematic strategy for collecting transcribed sequences and demonstrating their sites of expression is needed. It is necessarily an eventual goal of such a study to achieve closure; that is, to identify all mRNA species. Closure can be difficult to obtain due to the differing prevalence of various mRNA species and the large number of distinct mRNA species expressed by many distinct tissues. Nevertheless, the effort to achieve closure provides a progressively more reliable description of the dimensions of gene space.

Studies carried out in the laboratory of Craig Venter (M. D. Adams et al., “Complementary DNA Sequencing: Expressed Sequence Tags and Human Genome Project,” Science 252:1651-1656 (1991); M. D. Adams et al., “Sequence Identification of 2,375 Human Brain Genes,” Nature 355:632-634 (1992)) have resulted in the isolation of randomly chosen cDNA clones of human brain mRNA species, the determination of short single-pass sequences of their 3′-ends, about 300 base pairs, and a compilation of some 2500 of these as a database of “expressed sequence tags.” This database, while useful, fails to provide any knowledge of differential expression of RNAs in a tissue. It is therefore important to be able to recognize genes based on their overall pattern of expression within regions of brain and other tissues and in response to various paradigms, such as various physiological or pathological states or the effects of drug treatment, rather than simply their expression in a single tissue.

Other work has focused on the use of the polymerase chain reaction (PCR) to establish a database. Williams et al. (J. G. K. Williams et al., “DNA Polymorphisms Amplified by Arbitrary Primers Are Useful as Genetic Markers,” Nucl. Acids Res. 18:6531-6535 (1990)) and Welsh & McClelland (J. Welsh & McClelland, “Genomic Fingerprinting Using Arbitrarily Primed PCR and a Matrix of Pairwise Combinations of Primers,” Nucl. Acids Res. 18:7213-7218 (1990)) showed that single 10-mer primers of arbitrarily chosen sequences, i.e., any 10-mer primer off the shelf, when used for PCR with complex DNA templates such as human, plant, yeast, or bacterial genomic DNA, gave rise to an array of PCR products. The priming events were demonstrated to involve incomplete complementarity between the primer and the template DNA. Presumably, partially mismatched primer-binding sites are randomly distributed through the genome. Occasionally, two of these sites in opposing orientation were located closely enough together to give rise to a PCR product band. There were on average 8-10 products, which varied in size from about 0.4 to about 4 kb and had different mobilities for each primer. The array of PCR products exhibited differences among individuals of the same species. These authors proposed that the single arbitrary primers could be used to produce restriction fragment length polymorphism (RFLP)-like information for genetic studies. Others have applied this technology (S. R. Woodward et al., “Random Sequence Oligonucleotide Primers Detect Polymorphic DNA Products Which Segregate in Inbred Strains of Mice,” Mamm. Genome 3:73-78 (1992); J. H. Nadeau et al., “Multilocus Markers for Mouse Genome Analysis: PCR Amplification Based on Single Primers of Arbitrary Nucleotide Sequence,” Mamm. Genome 3:55-64 (1992)).

Two groups (J. Welsh et al., “Arbitrarily Primed PCR Fingerprinting of RNA,” Nucl. Acids Res. 20:4965-4970 (1992); P. Liang & A. B. Pardee, “Differential Display of Eukaryotic Messenger RNA by Means of the Polymerase Chain Reaction,” Science 257:967-971 (1992)) adapted the method to compare mRNA populations. In the study of Liang and Pardee, this method, called mRNA differential display, was used to compare the population of mRNA species expressed by two related cell types, normal and tumorigenic mouse A31 cells. For each experiment, they used one arbitrary 10-mer as the 5′-primer and an oligonucleotide complementary to a subset of poly A tails as a 3′ anchor primer, performing PCR amplification in the presence of ³⁵S-dNTPs on cDNAs prepared from the two cell types. The products were resolved on sequencing gels and 50-100 bands ranging from 100-500 nucleotides were observed. The bands presumably resulted from amplification of cDNAs corresponding to the 3′-ends of mRNA molecules that contain the complement of the 3′ anchor primer and a partially mismatched 5′ primer site, as had been observed on genomic DNA templates. For each primer pair, the pattern of bands amplified from the two cDNAs was similar, with the intensities of about 80% of the bands being indistinguishable. Some of the bands were more intense in one or the other of the PCR samples; a few were detected in only one of the two samples.

Further studies (P. Liang et al., “Distribution and Cloning of Eukaryotic mRNAs by Means of Differential Display: Refinements and Optimization,” Nucl. Acids Res. 21:3269-3275 (1993)) have demonstrated that the procedure works with low concentrations of input RNA (although it is not quantitative for rarer species), and the specificity resides primarily in the last nucleotide of the 3′ anchor primer. At least a third of identified differentially detected PCR products correspond to differentially expressed RNAs, with a false positive rate of at least 25%.

If all of the 50,000 to 100,000 mRNAs of the mammal were accessible to this arbitrary-primer PCR approach, then about eighty to ninety-five 5′ arbitrary primers and twelve 3′ anchor primers would be required in about 1000 PCR panels and gels to give a likelihood, calculated by the Poisson distribution, that about two-thirds of these mRNAs would be identified.

It is unlikely that all mRNA species are amenable to detection by this method for the following reasons. For a mRNA molecule to surface in such a survey, it must be prevalent enough to produce a signal on the autoradiograph and contain a sequence in its 3′ terminal 500 nucleotides capable of serving as a site for mismatched primer binding and priming. The more prevalent an individual mRNA species, the more likely it would be to generate a product. Thus, prevalent species may give bands with many different arbitrary primers. Because this latter property would contain an unpredictable element of chance based on selection of the arbitrary primers, it would be difficult to approach closure by the arbitrary primer method. Also, for the information to be portable from one laboratory to another and reliable, the mismatched priming must be highly reproducible under different laboratory conditions using different PCR machines, with the resulting slight variation in reaction conditions. As the basis for mismatched priming is poorly understood, this is a drawback of building a database from data obtained by the Liang & Pardee differential display method.

Sutcliffe, J. G., et al. in International published application PCT/US99/23655, U.S. Pat. No. 5,459,037, U.S. Pat. No. 5,807,680, U.S. Pat. No. 6,030,784, U.S. Pat. No. 6,096,503, and U.S. Pat. No. 6,110,680, all hereby incorporated by reference as part of this disclosure, describe an improved method of simultaneous sequence-specific identification of mRNA species known as TOGA™ (TOtal Gene expression Analysis), that eliminates the uncertain aspect of 5′-end generation and provides for closure. See also Sutcliffe, et al. Proc. Natl. Acad. Sci. USA, 97(5):1976-1981 (2000), also incorporated by reference. This TOGA method does not depend on potentially irreproducible mismatched priming, reduces the number of PCR panels and gels required for a complete survey, and allows double-strand sequence data to be rapidly accumulated.

The TOGA™ method provides methods and compositions useful for sequence-specific identification of mRNA species expressed in a tissue in terms of two characteristics: first, the length of a fragment comprising the 5′ end of the poly (A) tail and the next upstream recognition site of a chosen restriction endonuclease, and second, a partial sequence of the 5′ end of the fragment. The partial sequence of the 5′ end of the fragment includes the sequence of the recognition site of the chosen restriction endonuclease and two to six nucleotides adjacent and 3′ to the recognition site of the chosen restriction endonuclease. The methods further provide convenient means of comparing the results to entries in databases of sequences of polynucleotides and polypeptides.

There remains a need for further improvements of the original TOGA™ method as disclosed in the references cited above. We have refined the original TOGA™ technique to make it more reproducible, more sensitive, amenable to automation and easier to use.

### SUMMARY OF THE INVENTION

The present invention provides a simplified method, also called simplified TOGA™, for simultaneous sequence-specific identification of multiple mRNA molecules in a mRNA population without the necessity of making a library. In a preferred embodiment, the method comprises the steps of:

preparing a population of capturable double-stranded cDNA molecules from a population of mRNA molecules having a 3′ poly (A) terminus by using a mixture of anchor primers, each anchor primer having a 5′ terminus and a 3′ terminus and including: (i) phasing residues located at the 3′ terminus of each of the anchor primers selected from the group consisting of -V, -V-N, and -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N where the anchor primer phasing residues in the mixture are defined by one of -V, -V-N, or -V-N-N; (ii) a tract of 8 to 40 T residues located towards the 5′-terminus relative to the phasing residues; (iii) a first stuffer segment consisting of 4 to 40 nucleotides; (iv) a segment complementary to a 3′ PCR primer consisting of about 12 to about 20 nucleotide residues located towards the 5′-terminus relative to the tract of T residues; (v) a second stuffer segment consisting of 4 to 40 nucleotides and (vi) a capturable moiety affixed to the anchor primer;

digesting the population of capturable double-stranded cDNA molecules with a restriction endonuclease that recognizes at least a four nucleotide sequence not found within the sequence of the anchor primer, thereby producing a population of capturable double stranded cDNA fragments, each capturable double stranded cDNA fragment having an anchor end that corresponds to the poly(A) segment of the original mRNA molecule and including at least a portion of a sequence corresponding to that of the anchor primer, and a free end opposite to the anchor end;

capturing the capturable moiety, thereby affixing the capturable double-stranded cDNA fragments to a substrate to form affixed double stranded cDNA fragments;

ligating a double stranded adapter polynucleotide to the free end of each affixed double stranded cDNA fragment to form a population of adapted cDNA molecules, the double stranded adapter polynucleotide including a segment corresponding to the sequence of a bacteriophage RNA polymerase promoter and a segment complementary to a 5′ PCR primer;

generating a first set of sequence-specific PCR products by dividing the population of adapted cDNA molecules into a first series of subpools as templates for a first polymerase chain reaction with a 3′ PCR-primer about 15 to 30 nucleotides in length that is complementary to at least a portion of the anchor primer sequence and a first 5′ PCR-primer about 15 to about 30 nucleotides in length and that is complementary to a portion of the adapter polynucleotide, with the complementarity extending one nucleotide beyond the portion of the adapter polynucleotide into the specific sequence corresponding to the free end of the capturable cDNA and including a 3′-terminus consisting of-Nₓ, wherein X is an integer from 1 to 5, and N is selected from group consisting of the four deoxyribonucleotides A, C, G, and T, and wherein a different one of the first 5′ PCR primers is used in each of 4^{X} different subpools;

generating a detectable second set of sequence-specific PCR products by further dividing the first set of sequence-specific PCR products in each of the first series of subpools into a second series of subpools and using the first set of sequence-specific PCR products as templates for a second polymerase chain reaction with a 3′ PCR primer of 15 to 30 nucleotides in length comprising a detectable moiety and a second 5′ PCR primer defined as having a 3′-terminus consisting of -N_{X}-N_{X+Y,} wherein N_{X} is the same as the N_{X} used in the first polymerase chain reaction for that subpool, wherein Y is an integer from 1 to 5, (X+Y) is an integer from 2 to 6, N is selected from group consisting of the four deoxyribonucleotides A, C, G, and T, wherein the second 5′ PCR primer is about 15 to about 30 nucleotides in length and wherein the second 5′ PCR-primer is complementary to a portion of the adapter polynucleotide with the complementarity extending X+Y nucleotides beyond the portion of the adapter polynucleotide into the specific sequence corresponding to the free end of the capturable cDNA, wherein a different one of the second 5′ PCR primers is used in the different 4^{X+Y} subpools of the second series of subpools;

resolving the detectable second set of sequence-specific PCR products to generate a simultaneous display of sequence-specific PCR products representing the 3′-ends of mRNA species present in the mRNA population; and

characterizing each sequence-specific PCR product by a partial sequence and a length, thereby providing simultaneous sequence-specific identification of multiple mRNA molecules in a RNA population without making a cDNA library.

Typically, the method further comprises the steps of isolating RNA from a source and preparing a population of poly(A)-enriched mRNA molecules. Typically the RNA source is eukaryotic cells or tissue.

Typically, the capturable moiety is affixed to the 5′ end of the anchor primer oligonucleotide. In one preferred embodiment, the capturable moiety of the anchor primer is a biotin moiety affixed to the 5′ end of the anchor primer oligonucleotide.

Typically, the method further comprises the step of detecting the resolved second set of sequence-specific PCR products. Typically, the second set of sequence-specific PCR products is labeled by the incorporation of nucleotides labeled with a detectable moiety. In a preferred embodiment, the second set of sequence-specific PCR products is labeled with a fluorescent label, typically by the use of a PCR primer conjugated to a detectable moiety. The detectable moiety can be a radioisotope, a fluorescent label, a magnetic label or a chemical moiety such as biotin or digoxigenin. The detectable moiety can be detected directly, or indirectly, by the use of a labeled specific binding partner of the detectable moiety. Alternatively, the specific binding partner of the detectable moiety can be coupled to an enzymatic system that produces a detectable product.

In one preferred embodiment, the second set of sequence-specific PCR products is labeled by using a 3′ PCR primer conjugated to a fluorescent moiety and is detected by monitoring laser-induced fluorescent emission. In one preferred embodiment, the second set of sequence-specific PCR products is labeled by using a 3′ PCR primer conjugated to 3′,6′-dihydroxy-6-carboxyfluorescein (6-FAM).

In a preferred embodiment, the step of preparing a population of double-stranded cDNA molecules comprises the steps of synthesizing a first cDNA strand and synthesizing a second cDNA strand.

In a preferred embodiment, the method further comprises the steps of

producing synthetic RNA molecules using the adapted cDNA molecules as templates by incubating the adapted cDNA molecules with a bacteriophage RNA polymerase capable of initiating transcription from the sequence corresponding to the sequence of a bacteriophage RNA polymerase promoter; and

generating first-strand cDNA by transcribing the synthetic RNA using a reverse transcriptase and a RT primer that is 15 to 30 nucleotides in length and comprising a segment corresponding to a portion of the anchor primer sequence.

Typically, the anchor primer phasing residues are -V-N-N. In a preferred embodiment X=1 and (X+Y)=4. Typically, the tract of T residues comprises 18 T residues.

Typically, the anchor primer further comprises at least one segment comprising a sequence recognized by a restriction endonuclease that recognizes at least six bases, the segment being located towards the 5′-terminus relative to the anchor PCR primer segment. In one preferred embodiment, the anchor primer further comprises at least one segment comprising a sequence recognized by a restriction endonuclease that recognizes more than six bases, the segment being located towards the 5′-terminus relative to the anchor PCR primer segment. Preferred restriction endonucleases that recognize at least six bases are EcoRI and XbaI. Typically, the restriction endonuclease that recognizes more than six bases is selected from the group consisting of AscI, BaeI, FseI, NotI, PacI, PmeI, PUMI, RsrII, SapI, SexAI, SfiI, SgfI, SgrAI, SrfI, SseI and SwaI. A preferred restriction endonuclease that recognizes more than six bases is NotI.

In one embodiment, the anchor primer having a 5′ terminus and a 3′ terminus and includes: (i) anchor primer phasing residues located at the 3′ terminus of each of the anchor primers selected from the group consisting of -V, -V-N, and -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N where the anchor primer phasing residues in the mixture are defined by one of -V, -V-N, or -V-N-N; (ii) a tract of 8 to 40 T residues located towards the 5′-terminus relative to the phasing residues; (iii) a first stuffer segment consisting of 4 to 40 nucleotides 5′ relative to the tract of T residues; (iv) a segment complementary to a 3′ PCR primer consisting of about 12 to about 20 nucleotide residues located 5′ relative to the first stuffer segment; (v) a second stuffer segment consisting of 4 to 40 nucleotides located 5′ relative to the PCR primer segment; (vi) a restriction endonuclease site located 5′ to the PCR primer segment and (vii) a capturable moiety affixed to the 5′ terminus of the anchor primer.

In one preferred embodiment, the anchor primers have the sequence 5′-A-T-G-A-A-T-T-C-T-C-T-A-G-A-G-A-T-T-G-C-T-A-C-C-T-C-A-G-T-C-T-G-A-G-C-T-C-C-A-C-C-G-C-G-G-T-A-G-T-A-C-T-C-A-C-T-G-C-T-T-T-T-T-T-T-T-T-T-T-T-T-T-T-T-T-T-V-N-N-3′ (SEQ ID NO: 1) wherein the 5′ terminal base (base 1) is a biotinylated adenylate residue, V can represent A, C or G, and each N can represent A, C, G, or T. In more preferred embodiment, the anchor primers have the sequence 5′-A-T-G-A-A-T-T-C-T-C-T-A-G-A-G-T-C-T-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 2). In another more preferred embodiment, the anchor primers have the sequence 5′-G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 3).

In general, the bacteriophage RNA polymerase promoter is selected from the group consisting of T3 promoter, T7 promoter and SP6 promoter. In a preferred embodiment, the bacteriophage RNA polymerase promoter is a T3 promoter.

Typically, the restriction endonuclease recognizing a four-nucleotide sequence is selected from the group consisting of AciI, AluI, BfaI, BstUI, Csp6I, DpnI, DpnII, HaeIII, HhaI, HinP1I, HpaII, MaeII, MboI, MnlI, MseI, MspI, NlaIII, RsaI, Sau3AI. TaiI, TaqI, and Tsp509I. Preferred restriction endonucleases recognizing a four-nucleotide sequence include MspI, Sau3AI, NlaIII and TaqI. In one preferred embodiment, the restriction endonuclease recognizing a four-nucleotide sequence is Msp I.

In one preferred embodiment where the restriction endonuclease that recognizes at least a four nucleotide sequence not found within the sequence of the anchor primer is MspI or TaqI, one strand of the double stranded adapter polynucleotide comprises the sequence 5′-A-T-G-A-A-T-T-C-G-G-T-A-C-C-A-A-T-T-A-A-C-C-C-T-C-A-C-T-A-A-A-G-G-G-A-C-A-G-C-T-T-A-T-C-A-T-C-G-C-T-C-G-A-G-C-T-C-G-A-C-G-G-T-A-T-3′ (SEQ ID NO:7) and the other strand of the double stranded adapter polynucleotide comprises the sequence 5′-C-G-A-T-A-C-C-G-T-C-G-A-G-C-T-C-G-A-G-C-G-A-T-G-A-T-A-A-G-C-T-G-T-C-C-C-T-T-T-A-G-T-G-A-G-G-G-T-T-A-A-T-T-G-G-T-A-C-C-G-A-A-T-T-C-A-T-3′ (SEQ ID NO:8).

In another preferred embodiment, one strand of the double stranded adapter polynucleotide comprises the sequence 5′-Phospho-C-G-A-T-A-C-C-G-T-C-G-A-C-C-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:9), and the other strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-G-G-T-C-G-A-C-G-G-T-A-T-3′ (SEQ ID NO:10).

In another preferred embodiment where the restriction endonuclease that recognizes at least a four nucleotide sequence not found within the sequence of the anchor primer is Sau3AI, one strand of the double stranded adapter polynucleotide comprises the sequence 5′-Phospho-G-A-T-C-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:11), and the other strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-3′ (SEQ ID NO:12).

In another preferred embodiment where the restriction endonuclease that recognizes at least a four nucleotide sequence not found within the sequence of the anchor primer is NlaIII, one strand of the double stranded adapter polynucleotide comprises the sequence 5′-Phospho-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:13), and the other strand of the double stranded adapter polynucleotide comprises the 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-C-A-T-G-3′ (SEQ ID NO:14).

Suitable 3′ PCR primers are capable of hybridizing to the 3′ PCR correlate segment of the specific anchor primer oligonucleotide used. Suitable 3′ PCR primers are selected from the group consisting of 5′-G-A-G-C-T-C-C-A-C-C-G-C-G-G-T-3′ (SEQ ID NO: 5) and 5′-G-A-G-C-T-C-G-T-T-T-T-C-C-C-A-G-3′ (SEQ ID NO:6). In one preferred embodiment, the 3′ PCR primer comprises the sequence 5′-G-A-G-C-T-C-C-A-C-C-G-C-G-G-T-3′ (SEQ ID NO:5).

Typically, the reverse transcriptase (RT) No primer comprises the sequence 5′-C-A-G-T-C-T-G-A-G-C-T-C-C-A-C-C-G-C-G-G-T-3′ (SEQ ID NO: 15). In another embodiment, the reverse transcriptase (RT) No primer comprises the sequence 5′-G-A-G-C-T-C-C-A-C-C-G-C-G-G-T-3′ (SEQ ID NO:5).

In one preferred embodiment where the restriction endonuclease that recognizes at least a four nucleotide sequence not found within the sequence of the anchor primer is MspI, the first 5′ PCR N₁ primer comprises the sequence 5′-C-T-C-G-A-G-C-T-C-G-A-C-G-G-T-A-T-C-G-G-N-3′ (SEQ ID NO: 16). In another such preferred embodiment, the first 5′ PCR N₁ primer comprises the sequence 5′-C-C-T-C-G-A-G-G-T-C-G-A-C-G-G-T-A-T-C-G-G-N-3′ (SEQ ID NO: 17). Typically X=1 and Y=3, and in such a preferred embodiment, the second 5′ N_{X}-N_{X+Y} PCR primer comprises the sequence 5′-C-G-A-C-G-G-T-A-T-C-G-G-N-N-N-N-3′ (SEQ ID NO:18).

In one preferred embodiment where the restriction endonuclease that recognizes at least a four nucleotide sequence not found within the sequence of the anchor primer is Sau3AI, the first 5′ PCR N_{X} primer comprises the sequence 5′ A-G-C-T-C-T-G-T-G-G-T-G-A-G-G-A-T-C-N-3′ (SEQ ID NO: 19). In such a preferred embodiment, where X=1 and Y=3, the second 5′ N_{X}-N_{X+Y} PCR primer comprises the sequence 5′-C-T-C-T-G-T-G-G-T-G-A-G-G-A-T-C-N-N-N-N-3′ (SEQ ID NO:20).

In one preferred embodiment where the restriction endonuclease that recognizes at least a four nucleotide sequence not found within the sequence of the anchor primer is NlaIII, the first 5′ PCR N_{X} primer comprises the sequence 5′-A-G-C-T-C-T-G-T-G-G-T-G-A-G-C-A-T-G-N-3′ (SEQ ID NO:21). In such a preferred embodiment, where X=1 and Y=3, the second 5′ N_{X}-N_{X+Y} PCR primer comprises the sequence 5′-C-T-C-T-G-T-G-G-T-G-A-G-C-A-T-G-N-N-N-N-3′ (SEQ ID NO:22).

In one preferred embodiment, where the restriction endonuclease that recognizes at least a four nucleotide sequence not found within the sequence of the anchor primer is TaqI, the first 5′ PCR N_{X} primer comprises the sequence 5′-C-C-T-C-G-A-G-G-T-C-G-A-C-G-G-T-A-T-C-G-A-N -3′ (SEQ ID NO:23). In such a preferred embodiment, where X=1 and Y=3, the second 5′ N_{X}-N_{X+Y} PCR primer comprises the sequence 5′-T-C-G-A-G-G-T-C-G-A-C-G-G-T-A-T-C-G-A-N-N-N-N-3′ (SEQ ID NO:24).

In one preferred embodiment, the capturable moiety of the anchor primer of the double stranded DNA molecule is affixed to a substrate comprising a coating. Suitable coated substrates include microtitre plates, PCR tubes, polystyrene beads, silica beads, paramagnetic polymer beads and paramagnetic porous glass particles. A preferred coated substrate is a suspension of paramagnetic polymer beads (Dynal, Inc., Lake Success, N.Y.).

In one preferred embodiment, the capturable moiety of the anchor primer is a biotin moiety conjugated to the anchor primers, preferably to the 5′ terminus of the anchor primers. In such an embodiment, the first restricted cDNA is separated from the remainder of the cDNA by contacting the first restricted cDNA with a streptavidin-coated substrate. A preferred streptavidin-coated substrate is a suspension of paramagnetic polymer beads (Dynal, Inc., Lake Success, N.Y.).

In another preferred embodiment, the capturable moiety of the anchor primer of the double stranded DNA molecule is affixed to a substrate comprising a coating of N-oxysuccinimide ester.

In general, the capturable moiety of the anchor primer is affixed to a substrate comprising a coating selected from the group consisting of streptavidin, avidin, neutravidin, N-oxysuccinimide ester, dimethyladipimidate-2-HCl, dimethylpimelimidate-HCl, dimethylsuberimidate-2HCl, dimethyl 3,3′-dithiobisproprionimidate-2HCl, disuccinimidyl glutarate, disuccinimidyl suberate, bis(sulfosuccinimidyl)suberate, dithiobis(succinimidyl proprionate), dithiobis(sulfosuccinimidyl proprionate), ethylene glycobis(succinimidylsuccinate), ethylene glycobis(sulfosuccinimidylsuccinate), disuccinimidyl tartarate, disulfosuccinimidyl tartarate, bis[2-succinimidyloxycarbonyloxy)ethyl]sulfone, bis[2-(sulfosuccinimidyloxycarbonyloxy) ethyl]sulfone, and N-hydroxysuccinimidyl 2,3-dibromopropionate, succinimidyl 4-(N-maleimidomethyl) cyclohexane-l-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, succinimidyl 4-(p-maleimidophenyl)-butyrate, sulfosuccinimidyl 4-(p-maleimidophenyl)-butyrate, bismaleimidohexane, N-(γ-maleimidobutyryloxy)succinimide ester, N-(γ-maleimidobutyryloxy)sulfosuccinimide ester, N-succinimidyl(4-iodoacetyl)aminobenzoate, sulfosuccinimidyl(4-iodoacetyl)aminobenzoate, 1,4-Di-[3′-2′-pyridyldithio(propionamido)butane], 4-succinimidyloxycarbonyl-α-(2-pyridyldithio)toluene, sulfosuccinimidyl-6-[α-methyl-α-(2-pyridyldithio)-toluamido]hexane, N-succinimidyl-3-(2-pyridyldithio)-propionate, succinmidyl 6-[3-(2-pyridyldithio)propionamido]hexanoate, sulfosuccinmidyl 6-[3-(2-pyridyldithio)propionamido]hexanoate, 1-ethyl-3-(3-dimethylaminopropyl)carbodiimide hydrochloride, N,N′-dicyclohexylcarbodiimide, 1,5-difluoro-2,4-dinitrobenzene, N-5-azido-2-nitrobenzoyloxysuccinimide, N-hydroxysuccinimidyl-4-azidobenzoate, N-hydroxysulfosuccinimidyl-4-azidobenzoate, N-hydroxysuccinimidyl-4-azidosalicylic acid, N-hydroxysulfosuccinimidyl-4-azidosalicylic acid, sulfosuccinimidyl-(4-azidosalicylamido)-hexanoate, p-nitrophenyl-2-diazo-3,3,3-trifluoropropionate, 2-diazo-3,3,3-trifluoropropionylchloride, N-succinimidyl-(4-azidophenyl)1,3′-dithiopropionate, sulfosuccinimidyl-(4-azidophenyldithio)propionate, sulfosuccinimidyl 2-(7-azido-4-methylcoumarin-3-acetamide) ethyl-1,3′-dithiopropionate, sulfosuccinimidyl 7-azido-4-methylcoumarin-3-acetate, sulfosuccinimidyl 2(m-azido-o-nitrobenzamido)-ethyl-1,3′-dithiopropionate, N-succinimidyl-6-(4′-azido-2′-nitrophenylamino)hexanoate, sulfosuccinimidyl-6-(4′-azido-2′-nitrophenylamino)hexanoate, sulfosuccinimidyl 2-(p-azidosalicylamido)ethyl-1,3′-dithiopropionate, and sulfosuccinimidyl 4-(p-azidophenyl)butyrate and mixtures thereof.

The simplified method sorts mRNA species on the basis of an identity or address determined by 1) a partial nucleotide sequence of length a+b, where a is the length in bases of the restriction endonuclease recognition site and b is the number of parsing bases, where 6≧b≧3, and 2) the distance of that partial sequence from the poly(A) tail. Typically the identity or address is determined by a partial sequence that includes a four base recognition site for a restriction endonuclease and four parsing bases. In one preferred embodiment, the recognition site for a restriction endonuclease is MspI, and the partial sequence is C-C-G-G-N₁-N₂-N₃-N₄. Because it is dependent upon the nucleotide sequence of a mRNA species and not its prevalence in a given tissue, the method can account for all mRNA species present at concentrations above its detection threshold. In contrast to differential display and RAP-PCR methodologies, there is no uncertain aspect to the generation of 5′ ends of the PCR amplified products.

According to one preferred embodiment of the method of the present invention (FIG. 1), the populations of cDNA fragments produced from each of the mRNA samples contain copies of the extreme 3′ ends, from the most distal site for MspI to the beginning of the poly(A) tail, of nearly all poly(A)⁺ mRNA molecules in the starting RNA sample approximately according to the initial relative concentrations of the mRNA molecules. Because both ends of the cDNA fragments for each species are exactly defined by the sequence of the mRNA molecules themselves, the fragment lengths are uniform for each molecular species, allowing their later visualization as discrete bands on gels or as fragments in mass spectrometry. Messenger RNA species that lack MspI recognition sites are not represented, but these are relatively rare. These mRNA species can be analyzed by applying the method using a restriction endonuclease that recognizes a different four base recognition sequence.

Another aspect of such embodiments of the present invention is the use of sequences adjacent to the 3′ restriction endonuclease site, in one preferred embodiment, a MspI site, to sort the cDNAs in at least two successive PCR steps. The first PCR step utilizes a primer that anneals with sequences derived from the polynucleotide adapter ligated to the 5′ end of the cDNA fragment and extends into the cDNA fragment to include the first adjacent nucleotide (N₁) of the fragment. This step segregates the starting population of mRNA molecules into 4 subpools. In a second PCR step, each of the 4 subpools produced by the first PCR step is further segregated by division into 64 for a total of 256 subsubpools by using more insert-invasive primers, for example, having a 3′ terminus of four parsing bases, N₁N₂N₃N₄. A fluorescent label is incorporated into the products for their detection by laser-induced fluorescence by using fluorescent labeled 3′ PCR primers in the final PCR step. While using fluorescent labeled 3′ PCR primers in the final PCR step is a preferred means of labeling all species of PCR products, subsets of PCR products can be labeled using by using fluorescent labeled 5′PCR N₁N₂N₃N₄ primers in the final PCR step.

In a preferred embodiment, a separation technique such as electrophoresis is used to resolve the labeled molecules of the sequence-specific PCR products into distinct bands of measurable intensities and corresponding to measurable lengths. Suitable separation techniques include gel electrophoresis, capillary electrophoresis, HPLC, MALDI mass spectrometry and other suitable separations techniques known in the art that are capable of single base resolution are encompassed by the present invention.

In one preferred embodiment, each final PCR reaction product is thus assigned an identity or “digital address” based upon an 8-nucleotide sequence (when a restriction endonuclease recognizing a four base sequence is used and four parsing bases are used) including the four base restriction endonuclease site plus four parsing bases (e.g., C-C-G-G-N₁-N₂-N₃-N₄) and the distance of that sequence from the junction between the end of the message and the first A of the polyA tail at the 3′ end of the mRNA. When the nucleotide sequence of a PCR product fragment, either experimentally determined or determined from a database sequence, is known, the fragment is referred to as a digital sequence tag (DST): that is, a 3′-end EST (expressed sequence tag) derived by the method of the present invention. The intensity of the separated band of labeled PCR product fragments, detected using an appropriate method, preferably laser-induced fluorescence (alternatively, radioactive, magnetic labeling or mass spectroscopy may be used) is quantified and stored for each PCR product fragment in a database with the digital address (as defined herein) assigned for that PCR product fragment. The intensity of the separated band of labeled PCR product fragments is proportional to the starting amount of mRNA corresponding to that PCR product fragment.

In one embodiment, the three nucleotides at the 3′ end of the first 5′ PCR primer are joined by phosphodiesterase-resistant linkages, preferably phosphorothioate linkages.

Typically, the phasing residues of the anchor primers have a 3′ terminus of -V-N-N. In other embodiments, the phasing residues of the anchor primers have a 3′ terminus of -V or -V-N.

Typically, the RNA population has been enriched for polyadenylated mRNA species.

Typically, the resolving of the sequence-specific PCR products is conducted by electrophoresis to separate the PCR products. Preferably, the intensity of the laser-induced fluorescence of the resolved fluorescent labeled sequence-specific PCR products is about proportional to the abundances of the mRNA species corresponding to the sequence-specific PCR products in the original sample. In a preferred embodiment, the method further comprises a step of determining the relative abundance of each mRNA in the original sample from the intensity of the resolved fluorescent labeled sequence-specific PCR products corresponding to each mRNA species.

Typically, the step of resolving the sequence-specific PCR products by electrophoresis comprises electrophoresis of the fragments on multiple gels. In another embodiment, the step of resolving the sequence-specific PCR products by electrophoresis is performed using capillary electrophoresis. In another embodiment, the step of resolving the sequence-specific PCR products is performed by mass spectrometry.

In another embodiment, the present invention provides a data processing system for storing and displaying characteristics of polynucleotide fragments comprising, in combination, a graphical user interface for visually displaying characteristics of polynucleotide fragments, such as the sequence-specific PCR products, and at least one database for storing characteristics of polynucleotide fragments stored on a computer-readable medium. Typically, the database is constructed comprising the data produced by the quantitation of the fragment length and relative abundance of sequence-specific PCR products, including the characteristics of fragment length, relative abundance and partial sequence for each sequence-specific PCR product. Typically, the database further comprises data concerning sequence relationships, gene mapping and cellular distributions.

### BRIEF DESCRIPTION OF THE DRAWINGS

These and other features, aspects, and advantages of the present invention will become better understood with reference to the following description, appended claims, and accompanying drawings where:

FIG. 1 presents diagrammatically one preferred embodiment of the simplfied method of the present invention, showing the various steps of anchor primer alignment, synthesis of double stranded cDNA, restriction endonuclease digestion, capture, adapter ligation, synthetic RNA transcription, reverse transcription, and PCR steps using N₁ and N₄ primers, showing the sequences of anchor and other primers schematically—see text for details, including sequences.

FIG. 2 presents diagrammatically another preferred embodiment of the simplified method of the present invention, showing the various steps of anchor primer alignment, synthesis of double stranded cDNA, restriction endonuclease digestion, capture, adapter ligation, and PCR steps using N₁ and N₁-N₂-N₃-N₄ primers, showing the sequences of anchor and other primers schematically—see text for details, including sequences.

FIGS. 3A-D compares TOGA™ display profiles of PCR products generated using the TOGA™ method described in Example 1 and the simplified TOGA™ method of the present invention, where TOGA™ display profiles from serum starved (A and C) and serum replenished (B and D) MG63 human osteosarcoma cells in original TOGA™ (A and B) and simplified TOGA™ (C and D). Profiles were generated using second 5′ PCR N₁N₂N₃N₄ primers with parsing bases CCCG. Data are plotted as fluorescence intensity versus size in base pairs (75-500 b.p.). The fragment lengths in original TOGA™ are offset from simplified TOGA™ by +2bp.

FIGS. 4A-C show the results of simplified TOGA™ validation. FIGS. 4A-B show TOGA™ display profiles generated from serum starved (FIG. 4A) and serum replenished (FIG. 4B) MG63 human osteosarcoma cells using second 5′ PCR N₁N₂N₃N₄ primer with parsing bases CCCG. Data are plotted as fluorescence intensity versus size in base pairs (75-500 b.p.). The vertical guideline indicates a peak corresponding to virtual DST (i.e., having the expected digital address, length and partial sequence) of NF-κB (GenBank Accession No. M58603). The traces containing the indicated peaks are shown overlaid with traces generated using an 14 nucleotide extended 5′ PCR primer specific for NF-κB (5′-G-A-T-C-G-A-A-T-C-C- G-G-C-C-C-G-C-C-T-G-A-A-T-C-A-T-T-C-T-C-3′, SEQ ID NO:25). FIG. 4C shows a Northern blot prepared from RNA samples of serum starved (−) and serum replenished (+) MG63 cells. The blot was hybridized with human probes for NF-κB (based on the PCR product identified by the vertical guideline in the TOGA™ panel CCCG) and ribosomal protein S20. The lower band represents the mRNA for ribosomal protein S20, which was used as a normalization standard. Arrows indicate the relevant RNA bands.

FIGS. 5A-D provide a demonstration of the reproducibility of the simplified TOGA™ method of the present invention. TOGA™ display profiles of PCR products generated from serum starved (upper trace, OS−) and serum replenished (lower trace, OS+) MG63 human osteosarcoma cells using a second 5′ PCR N₁N₂N₃N₄ primer with parsing bases ACTC. Simplified TOGA™ was performed in duplicate using the alternative embodiment of Example 3 (FIGS. 5A-B) or the alternative embodiment of Example 4 (FIGS. 5C-D). Data from duplicate samples are shown overlaid in each panel.

### DETAILED DESCRIPTION OF PREFERRED EMBODIMENTS

We have developed a simplified TOGA™ method for simultaneous sequence-specific identification and display of mRNA molecules in RNA population. In one aspect, the method is useful for determining tissue-specific patterns of gene expression. In one aspect, the method is useful for determining mechanisms of drug action. In another aspect, the method is useful for drug screening. In another aspect, the method is useful for studying physiological processes. In a further aspect, the method provides diagnostic, prognostic and therapeutic reagents, kits and methods for pathological conditions. In yet another aspect, the method is useful for genomic mapping. The simplified method in its various applications provides several significant advantages over the presently available methods.

The simplified method of the present invention eliminates the time consuming step of producing a library. Most previous methods have required the cloning of the obtained DNA products in order to have sufficient quantities for reproducible PCR template production.

The elimination of the bacterial transformation step provides a number of useful advantages. The loss of sequences in the sample due to bacterial selection against certain sequences is eliminated. The potential for clone rearrangement during propagation is also removed. There is no selective loss of low copy number plasmids during plasmid DNA recovery. The potential for contamination with bacterial chromosomal DNA is abolished. Since the removal of the vector DNA after library construction is not required, the step of library clone linearization. In some embodiments, the step of cRNA synthesis can be eliminated.

The greater efficiency of ligation of adapters to cDNA compared to the ligation of cDNA to plasmid vector DNA is advantageous. Notably, the greater efficiency of ligation of adapters to cDNA results in more species of RNA represented in the final display.

As an important result of the above advantages, the simplified method of the present invention requires less input RNA than similar library-based methods. The ease of adapting the simplified method of the present invention to automation provides the significant advantages of increased reproducibility and high throughput.

I. Overview

The simplified method can begin by the isolation of RNA molecules, or the method may be practiced on a sample of mRNA molecules that has been previously isolated. In one preferred embodiment, the method comprises the steps of:

a. isolation of a population of RNA molecules;

b. synthesis of a population of first strand cDNA molecules from the isolated population of mRNA molecules using anchor primer oligonucleotides that bind to the beginning of the poly(A) tail;

c. synthesis of a second strand of cDNA resulting in a population of capturable double stranded cDNA molecules;

d. restriction of the population of cDNA molecules with a restriction endonuclease recognizing a four nucleotide sequence;

e. capture of the restricted double stranded cDNA molecules;

f. ligation of an adapter to the captured restriction fragments to form a population of adapted cDNA molecules;

g. transcription of synthetic RNA molecules from the adapted cDNA molecules;

h. synthesis of cDNA molecules from the synthetic RNA molecules;

i. performance of a first polymerase chain reaction using a N_{X} 5′ PCR primer;

j. performance of a second polymerase chain reaction using a N_{X+Y} 5′ PCR primer; and

k. resolution of the products of the second polymerase chain reaction.

Typically, preferred embodiments further comprise the step of formation of a database of characteristics of the products of the second polymerase chain reaction, including length, partial sequence and the relative concentration of the corresponding mRNA in a sample. Typically, preferred embodiments further comprise the step of comparison of at least one characteristic of the products of the second polymerase chain reaction to at least one corresponding characteristic of a reference database.

Typically, a method for detecting a change in the pattern of mRNA expression in a tissue associated with a physiological or pathological change comprising the steps of:

obtaining a first sample of normal or neoplastic tissue that is not subject to the physiological or pathological change;

isolating a mRNA population from the first sample;

determining the pattern of mRNA expression in the first sample of the tissue by performing the general method to generate a first display of sequence-specific products representing the 3′-ends of mRNA species present in the first sample;

obtaining a second sample of the tissue that has been subject to the physiological or pathological change;

isolating a mRNA population from the second sample;

determining the pattern of mRNA expression in the second sample of the tissue by performing the general method to generate a second display of sequence-specific products representing the 3′-ends of mRNA species present in the second sample; and

comparing multiple displays to determine the effect of the physiological or pathological change on the pattern of mRNA expression in the tissue.

Typically more than two samples are compared. In preferred embodiments three, or more preferably at least four, samples are taken at multiple times or from multiple samples and compared.

The samples can be taken from an eukaryotic cell source. In one preferred embodiment, the samples are taken from animal tissue or from cultured animal cells. In another preferred embodiment, the samples are taken from plant tissue or from cultured plant cells.

Typically, the physiological or pathological change is selected from the group consisting of Alzheimer's disease, parkinsonism, ischemia, alcohol addiction, drug addiction, schizophrenia, amyotrophic lateral sclerosis, multiple sclerosis, depression, and bipolar manic-depressive disorder.

Typically, the physiological or pathological change is associated with learning or memory, emotion, glutamate neurotoxicity, feeding behavior, olfaction, vision, movement disorders, viral infection, electroshock therapy, the administration of a drug or the toxic side effects of drugs. Typically, the physiological or pathological change is selected from the group consisting of circadian variation, aging, and long term potentiation. In general, the physiological or pathological change is selected from processes mediated by transcription factors, intracellular second messengers, hormones, neurotransmitters, growth factors and neuromodulators. Alternatively, the physiological or pathological change is selected from processes mediated by cell-cell contact, cell-substrate contact, cell-extracellular matrix contact and contact between cell membranes and cytoskeleton.

Preferably, the normal or neoplastic tissue comprises cells taken or derived from an organ or organ system selected from the group consisting of the cardiovascular system, the lymphatic system, the respiratory system, the digestive system, the peripheral nervous system, the central nervous system, the enteric nervous system, the endocrine system, the integument (including skin, hair and nails), the skeletal system (including bone and muscle), the urinary system and the reproductive system.

In preferred embodiments, the normal or neoplastic tissue comprises cells taken or derived from the group consisting of epithelia, endothelia, mucosa, glands, blood, lymph, connective tissue, cartilage, bone, smooth muscle, skeletal muscle, cardiac muscle, neurons, glial cells, spleen, thymus, pituitary, thyroid, parathyroid, adrenal cortex, adrenal medulla, adrenal cortex, pineal, skin, hair, nails, teeth, liver, pancreas, lung, kidney, bladder, ureter, breast, ovary, uterus, vagina, testes, prostate, penis, eye and ear.

Typically, the normal or neoplastic tissue is derived from a structure within the central nervous system selected from the group consisting of retina, cerebral cortex, olfactory bulb, thalamus, hypothalamus, anterior pituitary, posterior pituitary, hippocampus, nucleus accumbens, amygdala, striatum, cerebellum, brain stem, suprachiasmatic nucleus, and spinal cord.

The method of the present invention is also suitably applied to determination of genes expressed in eukaryotic cells of plant origin. See, generally, Leaver, C. J., Differential Gene Expression and Plant Development, Cambridge University Press, 1988.

RNA samples thus can also be obtained from any plant including angiosperms, gymnosperms, monocotyledons, and dicotyledons. Plants of interest include cereals; fruits, vegetables, woody species, and ornamental flowers. In plants the physiologically change is typically associated with disease resistance, such as resistance to a virus, bacterium, or fungus, insect resistance, herbicide resistance, or ripening. The physiological change can also be associated with levels of hormones, glycoalkaloids, sugar, starch, vitamins, or minerals. The physiological change can also be associated with the expression of foreign genes.

Preferably, the tissue comprises cells taken or derived from a plant tissue including a leaf, node, root, stem, epidermis, periderm, xylem, phloem, parenchyma, collenchyma, sclerenchyma, cortex, pith, flower, pollen, seed, fruit, or tumor tissue such as crown galls.

Plant cell types can include cells such as parenchyma cells, guard cells, trichomes, sclerenchyma cells, tracheids, vessel members, sieve cells, sieve tube members, albuminous cells, companion cells, collenchyma cells, fibers, and sclereids. A plant sample can be obtained from cultured plant cell lines, protoplasts, or tissues and aggregations of plant cells in culture, such as embryos or calluses. Examples of plant cells lines include, for example, ATCC 40100 (corn), ATCC 40316 (potato), ATCC 54000 (sugarcane), ATCC 54002 (tomato), ATCC 54011 (rice), ATCC 54017 (wheat), ATCC 54040 (tobacco), ATCC 54041 (grape), and ATCC 75817 (yew).

Typically, a method of detecting a difference in action of a drug to be screened and a known compound comprising the steps of-

(a) obtaining a first sample of tissue from an organism treated with a compound of known physiological function;

(b) isolating a mRNA population from the first sample;

(c) determining the pattern of mRNA expression in the first sample of the tissue by performing the general method to generate a first display of sequence-specific products representing the 3′-ends of mRNA species present in the first sample;

(d) obtaining a second sample of tissue from an organism treated with a drug to be screened for a difference in action of the drug and the known compound;

(e) isolating a mRNA population from the second sample;

(f) determining the pattern of mRNA expression in the second sample of the tissue by performing the general method to generate a second display of sequence-specific products representing the 3′-ends of mRNA species present in the second sample; and

(g) comparing the first and second displays in order to detect the presence of mRNA species whose expression is not affected by the known compound but is affected by the drug to be screened, thereby indicating a difference in action of the drug to be screened and the known compound.

Typically, the drug to be screened is selected from the group consisting of antidepressants, neuroleptics, tranquilizers, anticonvulsants, monoamine oxidase inhibitors, stimulants, anti-parkinsonism agents, skeletal muscle relaxants, analgesics, local anesthetics, cholinergics, antiviral agents, antispasmodics, steroids, and non-steroidal anti-inflammatory drugs.

More generally, the terms “drug to be screened” and “drug to be tested” are used herein to refer to a broad class of useful chemical and therapeutic agents including physiologically active steroids, antibiotics, antifungal agents, antibacterial agents, antineoplastic agents, analgesics and analgesic combinations, anorexics, anthelmintics, antiarthritics, antiasthia agents, anticonvulsants, antidepressants, antidiabetic agents, antidiarrheals, antihistamines, anti-inflammatory agents, antimigraine preparations, antimotion sickness preparations, antinauseants, antiparkinsonism drugs, antipruritics, antipsychotics, antipyretics, antispasmodics, including gastrointestinal and urinary; anticholinergics, sympathomimetics, xanthine derivatives, cardiovascular preparations including calcium channel blockers, betablockers, antiarrhythmics, antihypertensives diuretics, vasodilators including general, coronary, peripheral and cerebral; central nervous system stimulants, cough and cold preparations, decongestants, hormones, hypnotics, immunosuppressives, muscle relaxants, parasympatholytics, parasympathomimetics, psychostimulants, sedatives, tranquilizers, allergens, antihistaminic agents, anti-inflammatory agents, physiologically active peptides and proteins, ultraviolet screening agents, perfumes, insect repellents, hair dyes, and the like. The term “physiologically active” in describing the agents contemplated herein is used in a broad sense to comprehend not only agents having a direct pharmacological effect on the host but also those having an indirect or observable effect which is useful in the medical arts, e.g., the coloring or opacifying of tissue for diagnostic purposes, the screening of ultraviolet radiation from the tissues and the like.

For instance, typical fungistatic and fungicidal agents include thiabendazole, chloroxine, amphotericin, candicidin, fungimycin, nystatin, chlordantoin, clotrimazole, ethonam nitrate, miconazole nitrate, pyrrolnitrin, salicylic acid, fezatione, ticlatone, tolnaftate, triacetin, zinc, pyrithione and sodium pyrithione.

Steroids include cortisone, cortodoxone, fluoracetonide, fludrocortisone, difluorsone diacetate, flurandrenolone acetonide, medrysone, amcinafel, amcinafide, betamethasone and its esters, chloroprednisone, clorcortelone, descinolone, desonide, dexamethasone, dichlorisone, difluprednate, flucloronide, flumethasone, flunisolide, fluocinonide, flucortolone, fluoromethalone, fluperolone, fluprednisolone, meprednisone, methylmeprednisone, paramethasone, prednisolone and predisone.

Antibacterial agents include sulfonamides, penicillins, cephalosporins, penicillinase, erythromycins, linomycins, vancomycins, tetracyclines, chloramphenicols, streptomycins, and the like. Specific examples of antibacterials include erythromycin, erythromycin ethyl carbonate, erythromycin estolate, erythromycin glucepate, erythromycin ethylsuccinate, erythromycin lactobionate, lincomycin, clindamycin, tetracycline, chlortetracycline, demeclocycline, doxycycline, methacycline, oxytetracycline, minocycline, and the like.

Peptides and proteins include, in particular, small to medium-sized peptides, e.g., insulin, vasopressin, oxytocin, growth factors, cytokines as well as larger proteins such as human growth hormone.

Other agents encompass a variety of therapeutic agents such as the xanthines, triamterene and theophylline, the antitumor agents, 5-fluorouridinedeoxyriboside, 6-mercaptopurinedeoxyriboside, vidarabine, the narcotic analgesics, hydromorphone, cyclazine, pentazocine, bupomorphine, the compounds containing organic anions, heparin, prostaglandins and prostaglandin-like compounds, cromolyn sodium, carbenoxolone, the polyhydroxylic compounds, dopamine, dobutamine, 1-dopa, a-methyldopa, angiotensin antagonists, polypeptides such as bradykinin, insulin, adrenocorticotrophic hormone (ACTH), enkephalins, endorphins, somatostatin, secretin and miscellaneous compounds such as tetracyclines, bromocriptine, lidocaine, cimetidine or any related compounds.

Other agents include iododeoxyuridine, podophyllin, theophylline, isoproterenol, triamcinolone acetonide, hydrocortisone, indomethacin, phenylbutazone paraaminobenzoic acid, aminopropionitrile and penicillamine.

The foregoing list is by no means intended to be exhaustive, and any physiologically active agent may be tested by the method of the present invention.

II. Definitions

The “adapter molecule” as that term is used herein refers to a double stranded polynucleotide.

As used herein, unless otherwise specified, the term “polynucleotide” is defined to encompass DNA and RNA of both synthetic and natural origin. The polynucleotide may exist as single or double stranded DNA or RNA, or an RNA/DNA heteroduplex. Thus, the polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the term “oligonucleotide” refers to a single stranded DNA or RNA molecule. An “oligonucleotide primer” refers to an oligonucleotide that is hybridized to a nucleic acid template for sequencing purposes or to prime enzymatic synthesis of a second nucleic acid strand.

The “capturable anchor primer” as that term is used herein refers to the modified polydT primer used to generate the second strand nucleic acid from the template strand nucleic acid. The capturable anchor primer comprises a single stranded oligonucleotide sequence wherein a polydT sequence is found at the 3′ end and a capturable moiety is found at the 5′ end of the oligonucleotide.

The “capturable moiety” as that term is used refers to any functional group or molecule that can be joined to a nucleotide and can be attached to a substrate or solid-phase support. The capturable moiety can be directly or indirectly attached to a nucleotide and can also be directly or indirectly attached to a substrate or solid-phase support.

The term “spacer” is intended to encompass any suitable means that can be used to link a nucleotide of the anchor primer to the capturable moiety or any suitable means by which to link the capturable moiety to the solid-phase support. The spacer should not adversely affect the function of the anchor primer or the capturable moiety.

As used herein, the term “substrate” or “solid-phase support” is defined as any material having a rigid or semi-rigid surface. The substrate or solid support should have a surface chemistry that allows the substrate to form a direct attachment with a capturable moiety. Alternatively, the substrate or solid support should have a surface that can be coated or derivatized such that, upon coating or derivatization, the substrate can form an attachment with a capturable moiety. Suitable coated substrates include microtitre plates, PCR tubes, polystyrene beads, paramagnetic polymer beads and paramagnetic porous glass particles. A preferred coated substrate is a suspension of paramagnetic polymer beads (Dynal, Inc., Lake Success, N.Y.).

As used herein, the term “RNA polymerase promoter site” refers to the consensus sequence of the promoter site to which an RNA polymerase binds to initiate RNA synthesis.

As used herein, the term Digital Sequence Tags (DSTs), refers to polynucleotides that are expressed sequence tags of the 3′ end of mRNA molecules that have been characterized by an “digital address” consisting of a partial sequence and a length. The digital address provides a means of comparing DSTs to DSTs obtained in the TOGA analysis of other samples and to known sequences in appropriately transformed databases.

III. Current Embodiments

The first stage of the present invention involves the generation of a population of double stranded cDNA molecules wherein each cDNA molecule has a capturable anchor primer at the 3′ end. Such a population of double stranded cDNA molecules may be produced from a population of RNA molecules using methods well-known in the art. The population of RNA molecules used can be isolated from any eukaryotic tissue or cell population, including cells grown in culture. Preferably, the RNA molecules are isolated from tissue or cells actively transcribing a potential gene of interest. Methods of extraction of RNA are well-known in the art and are described, for example, in Sambrook et al., Molecular Cloning: A Laboratory Manual (Cold Spring Harbor Press, Cold Spring Harbor, N.Y., 1989), vol. 1, ch.7, “Extraction, Purification, and Analysis of Messenger RNA from Eukaryotic Cells”, incorporated herein by this reference. Other isolation and extraction methods are also well-known.

A. RNA Isolation

Typically, RNA isolation is performed in the presence of chaotropic agents such as guanidinium chloride or guanidinium thiocyanate, although other detergents and extraction agents can alternatively be used. The mRNA, i.e. poly(A)⁺ RNA, is typically isolated from the total extracted RNA by chromatography over oligo(dT)-cellulose or other chromatographic media that have the capacity to bind the polyadenylated 3′-end portion of mRNA molecules. Preferably poly(A)⁺ selected mRNA is used. Also preferably, the poly(A)⁺ selected mRNA is representative for all the expressed genes in the prepared sample. Typically, about 20 ng to about 2 μg of mRNA is used to prepare the population of double stranded cDNA molecules. Alternatively, about 1 μg to about 100 μg of total RNA may be used to synthesize cDNA.

B. First Strand cDNA Synthesis

1. Anchor Primers

The method of the present invention uses a modified poly(dT) primer that is a capturable anchor primer, using well-known methods. The first strand of cDNA is synthesized through the process of reverse transcription whereby the enzyme reverse transcriptase adds deoxyribonucleotides to the 3′ terminus of the poly(dT) primer (Varmus, Science, 240, 1427-1435 (1988); Sambrook et al., Molecular Cloning: A Laboratory Manual, vol. 2, “Construction and Analysis of cDNA Libraries”). The second strand cDNA is generated using an RNAse to cleave the RNA strand of the RNA:cDNA hybrid and a DNA polymerase to synthesize a complementary DNA strand from the remaining template DNA strand.

In general, each anchor primer having a 5′ terminus and a 3′ terminus and including: (i) phasing residues located at the 3′ terminus of each of the anchor primers selected from the group consisting of -V, -V-N, and -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N where the phasing residues in the mixture are defined by one of -V, -V-N, or -V-N-N; (ii) a tract of 8 to 40 T residues located towards the 5′-terminus relative to the phasing residues; (iii) a first stuffer segment consisting of 4 to 40 nucleotides; (iv) a segment complementary to a 3′ PCR primer consisting of about 12 to about 20 nucleotide residues located towards the 5′-terminus relative to the tract of T residues; (v) a second stuffer segment consisting of 4 to 40 nucleotides and (vi) a capturable moiety affixed to the anchor primer. In general, the nucleotide sequence comprising the anchor primer is chosen to avoid the formation of internal structures such as hairpins, and be rare in the genome being studied.

In a preferred embodiment, the anchor primer further comprises a recognition site for a restriction endonuclease. Preferably the recognition site for the restriction endonuclease comprises at least six nucleotides.

In some preferred embodiments, a first stuffer segment is interposed between the PCR primer site and the oligo (d)T tract. Typically, when present, the first stuffer segment comprises about six to about 24 nucleotides, preferably about eight to about 20 nucleotides, more preferably about ten to about 18 nucleotides. In one preferred embodiment the first stuffer segment comprises twelve nucleotides. In one preferred embodiment, the first stuffer segment has the nucleotide sequence A-G-T-A-C-T-C-A-C-T-G-C (SEQ ID NO:26). In another preferred embodiment, the first stuffer segment has the nucleotide sequence A-G-T-A-C-T-C-A-C-T-G-C-A-G (SEQ ID NO:27). In another embodiment, the first stuffer segment comprises a functional site chosen from the group consisting of a restriction endonuclease site, a PCR primer site and a RNA polymerase binding site. In those embodiments in which the first stuffer segment comprises a RNA polymerase binding site, the RNA polymerase binding site is different from the RNA polymerase binding site present in the adapter primer.

In some preferred embodiments, a second stuffer segment is interposed between the recognition site for a restriction endonuclease and the PCR primer site. Typically, when present, the second stuffer segment comprises about 4 to about 40 nucleotides, more preferably about ten to about 18 nucleotides. In one preferred embodiment the second segment comprises seventeen nucleotides. In another embodiment, the second stuffer segment comprises a functional site chosen from the group consisting of a restriction endonuclease site, a PCR primer site and a RNA polymerase binding site. In those embodiments in which the second stuffer segment comprises a RNA polymerase binding site, the RNA polymerase binding site is different from the RNA polymerase binding site present in the adapter primer.

In one preferred embodiment, the second stuffer segment has the nucleotide sequence 5′-G-A-T-T-G-C-T-A-C-C-T-C-A-G-T-C-T-3′ (SEQ ID NO:28).

The oligo dT tract of the anchor primer comprises at least 8 thymidine nucleotides, typically 8 to 40 thymidine nucleotides. Preferably, the oligo dT tract comprises about 10 to about 40 thymidine nucleotides. More preferably, the oligo dT tract comprises about 12 to about 25 thymidine nucleotides. Most preferably, the oligo dT tract comprises about 15 to about 30 thymidine nucleotides. In one preferred embodiment the oligo dT tract comprises 18 thymidine nucleotides.

In one preferred embodiment, the parsing bases are -V-V-N, and a set of anchor primers consists of 48 different anchor primers. Each member of this mixture of 48 primers initiates synthesis at a fixed position at the 3′ end of all copies of each mRNA species in the sample, thereby defining a 3′ endpoint for each species. In one preferred embodiment, the anchor primers have the sequence 5′-A-T-G-A-A-T-T-C-T-C-T-A-G-A-G-A-T-T-G-C-T-A-C-C-T-C-A-G-T-C-T-G-A-G-C-T-C-C-A-C-C-G-C-G-G-T-A-G-T-A-C-T-C-A-C-T-G-C-T-T-T-T-T-T-T-T-T-T-T-T-T-T-T-T-T-T-V-N-N-3′ (SEQ ID NO: 1) wherein the 5′ terminal base (base 1) is a biotinylated adenylate residue, V can represent A, C or G, and each N can represent A, C, G, or T. In more preferred embodiment, the anchor primers have the sequence 5′-A-T-G-A-A-T-T-C-T-C-T-A-G-A-G-T-C-T-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 2). In another more preferred embodiment, the anchor primers have the sequence 5′-G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 3).

2. Capturable Moieties

The capturable moiety can be any functional group or molecule that, first, can be joined to a nucleotide N and, second, can be captured by a substrate or solid-phase support, such that N remains affixed to the solid-phase support by the attachment of the capturable moiety. While the capturable moiety may form any type of attachment with the substrate or solid-phase support, the attachment must be of sufficient strength to maintain the nucleotide N on the solid-phase support. Preferably, the capturable moiety is attached to the substrate or solid-phase support by affinity attachment or by covalent bonding. In accordance with the present invention, suitable capturable moieties include, for example, avidin, streptavidin, neutravidin, biotin, primary amines, primary carboxylates, thiol alcohols, thiol carboxylates, carbonyls, sugars, lipids, and peptides.

In one preferred embodiment, the capturable moiety is a biotin molecule. Biotin is a vitamin that forms an affinity attachment with the avidin family of proteins. The biotin-avidin attachment is the strongest non-covalent attachment known, having an association constant of 10¹⁵ M⁻¹. In another equally preferred embodiment, the capturable moiety is a primary amine. A primary amine allows covalent attachment, for example, to an N-oxysuccinimide ester (NOS) by displacing the N-oxysuccinimide group, resulting in the formation of a very specific covalent bond.

In addition, amines can be attached to several other molecules useful for capture onto solid-phase supports, such as those described in U.S. Pat. No. 5,677,276, which is incorporated herein by reference. Other molecules that react with amines are known to the skilled artisan and include, for example, imidoesters and N-hydroxysuccinimidyl esters (NHS-esters). Imidoesters react with primary amines, resulting in an amidine bond (Staros et al., J. Biol. Chem., 256:5890-5893 (1981); Browne et al., Biochem. Biophys. Res. Comm., 67:126-132 (1975)). Examples of suitable imidoesters include dimethyladipimidate-2-HCl (Pierce #20664), dimethylpimelimidate-HCl (Pierce #20666), dimethylsuberimidate-2HCl (Pierce #20668), dimethyl 3,3′-dithiobisproprionimidate-2HCl (Pierce #20665).

NHS-esters react with primary and secondary amines, resulting in a covalent amide bond. Examples of suitable NHS-esters include disuccinimidyl glutarate (Pierce #29592), disuccinimidyl suberate (Pierce #21555), bis(sulfosuccinimidyl)suberate (Pierce #21579), dithiobis(succinimidyl proprionate) (Pierce #22585), dithiobis(sulfosuccinimidyl proprionate) (Pierce #21577), ethylene glycobis(succinimidylsuccinate) (Pierce #21565), ethylene glycobis(sulfosuccinimidylsuccinate) (Pierce #21566), disuccinimidyl tartarate (Pierce #20590), disulfosuccinimidyl tartarate (Pierce #20591), bis[2-(succinimidyloxycarbonyloxy)ethyl]sulfone (Pierce # 21554), bis[2-(sulfosuccinimidyloxycarbonyloxy)ethyl]sulfone (Pierce # 21556), and N-hydroxysuccinimidyl 2,3-dibromopropionate (Pierce #22340).

Other NHS-esters include NHS-ester maleimide componds. Suitable NHS-ester maleimide compounds include, for example, succinimidyl 4-(N-maleimidomethyl) cyclohexane-1-carboxylate (Pierce #22320), sulfosuccinimidyl 4-(N-maleimidomethyl) cyclohexane-1-carboxylate (Pierce #22322), m-maleimidobenzoyl-N-hydroxysuccinimide ester (Pierce #22310), m-maleimidobenzoyl-N-hydroxysulfosuccinimide ester (Pierce #22312), succinimidyl 4-(p-maleimidophenyl)-butyrate (Pierce #22315), sulfosuccinimidyl 4-(p-maleimidophenyl)-butyrate (Pierce #22317), bismaleimidohexane (Pierce #22319), N-(γ-maleimidobutyryloxy)succinimide ester (Pierce #22314), N-(γ-maleimidobutyryloxy)sulfosuccinimide ester (Pierce #22324).

Still other compounds that are reactive with primary amines are known in the art and include NHS-ester haloacetyls, NHS-ester pyridyl disulfides, carbodiimides, arylhalides and arylazides. Suitable NHS-ester haloacetyls include N-succinimidyl(4-iodoacetyl)aminobenzoate (Pierce #22325, 22326) and sulfosuccinimidyl(4-iodoacetyl)aminobenzoate (Pierce #22327, 22328). Suitable NHS-ester pyridyl disulfides include 1,4-Di-[3′-2′-pyridyldithio(propionamido)butane] (Pierce #21701), 4-succinimidyloxycarbonyl-α-(2-pyridyldithio)toluene (Pierce #21558, 21458), sulfosuccinimidyl-6-[α-methyl-α-(2-pyridyldithio)-toluamido]hexane (Pierce #21568, 21569), N-succinimidyl-3-(2-pyridyldithio)-propionate (Pierce #21557, 21657, 21757), succinmidyl 6-[3-(2-pyridyldithio)propionamido]hexanoate (Pierce #21651, 21652), and sulfosuccinmidyl 6-[3-(2-pyridyldithio)propionamido]hexanoate (Pierce #21651, 21652). Carbodiimides react with primary amines to form amide or hydrazone bonds. Examples of suitable carbodiimides are 1-ethyl-3-(3-dimethylaminopropyl)carbodiimide hydrochloride (Pierce #22980, 22981) and N,N′-dicyclohexylcarbodiimide (Pierce #20320). One example of a suitable arylhalide is 1,5-difluoro-2,4-dinitrobenzene (Pierce #21524).

As stated, several arylazides are reactive with amines, including N-5-azido-2-nitrobenzoyloxysuccinimide (Pierce #21551), N-hydroxysuccinimidyl-4-azidobenzoate (Pierce #21560), N-hydroxysulfosuccinimidyl-4-azidobenzoate (Pierce #21561), N-hydroxysuccinimidyl-4-azidosalicylic acid (Pierce #27715), N-hydroxysulfosuccinimidyl-4-azidosalicylic acid (Pierce #27725), sulfosuccinimidyl-(4-azidosalicylamido)-hexanoate (Pierce #27735), p-nitrophenyl-2-diazo-3,3,3-trifluoropropionate (Pierce #20669), 2-diazo-3,3,3-trifluoropropionylchloride (Pierce #20669), N-succinimidyl-(4-azidophenyl)1,3′-dithiopropionate (Pierce #21552), sulfosuccinimidyl-(4-azidophenyldithio)propionate (Pierce #21553), sulfosuccinimidyl 2-(7-azido-4-methylcoumarin-3-acetamide) ethyl-1,3′-dithiopropionate (Pierce #33030), sulfosuccinimidyl 7-azido-4-methylcoumarin-3-acetate (Pierce #33025), sulfosuccinimidyl 2(m-azido-o-nitrobenzamido)-ethyl-1,3′-dithiopropionate (Pierce #21549), N-succinimidyl-6-(4′-azido-2′-nitrophenylamino)hexanoate (Pierce #22588), sulfosuccinimidyl-6-(4′-azido-2′-nitrophenylamino)hexanoate (Pierce #22589), sulfosuccinimidyl 2-(p-azidosalicylamido)ethyl-1,3′-dithiopropionate (Pierce #27716), and sulfosuccinimidyl 4-(p-azidophenyl)butyrate (Pierce #21562).

Other capturable moieties, such as sugar groups and thiol groups, are known in the art. Compounds reactive with sugar groups, such as phenyl azide-hydrazide, can be useful for capture onto solid-phase supports. Compounds reactive with thiol groups include maleimides, haloacetyls and pyridyl disulfides, such as the NHS-ester maleimide, NHS-ester haloacetyl, and NHS-ester pyridyl disulfide compounds provided above. Still other possible capturable moieties include non-selective photoreactive compounds, such as azidobenzoyl Hydrazide Pierce #21510, 21509), N-[4-azidosalicylamindo)butyl]-3′ (2′-pyridyldithio)propionamide (Pierce 21512), p-Azidophenylglyoxal monohydrate, 4-(p-Azidosalicylamido)butylamine, 1-(p-Azidosalicylamido)-4-(iodoacetamido)butane (Pierce #21511), and Bis-[L-4-azidosalicylamido)ethyl]disulfide (Pierce #21564).

The capturable moiety may be attached to an anchor primer using methods well-known in the art (see Hermannson, Bioconjugate Techniques (Academic Press, New York, (1996); Wong, S., Chemistry of Protein Conjugation and Cross-linking (CRC Press, Florida (1991)). For example, reagents for incorporating a primary amine into oligonucleotides are commercially available (Clontech, #5203-1, #5207-2, #5202-1) and described in the art (Connolly, B., Nuc. Acid Res., 15:3131 (1987); Agrawal et al., Nuc. Acid Res., 14:6227 (1986); Smith et al., Nuc. Acid Res., 12:2399 (1985); Sproat et al., Nuc. Acid Res., 15:6181 (1987); Sinha et al., Nuc. Acid Res., 16:2659 (1988)). Also, reagents for incorporating other capturable moieties, such as a biotin molecule or a thiol functional group, into oligonucleotides are commercially available (Clontech, #5024-1, #5021-1, #5211-1). For example, biotinylated UTPs, which are commercially available with different sized linkers (i.e. Biotin-11-dUTP, Enzo Biochemicals; Biotin-21-UTP, Clontech; Biotin-16-UTP, Boehringer Mannheim) can be used to produce biotinylated nucleic acids (Ampliscribe T7 High Transcription Kit, Epicentre). One skilled in the art would realize additional methods and reagents for incorporating capturable moieties into oligonucleotides, such as, for example, introducing a thiol group into an amine-modified oligonucleotide through reaction with the amine group using 2-iminothiolane or Traut's reagent (Pierce #26101), SATA (Pierce #26102), or SPDP (Pierce #21757, 21657, 21557).

Additionally, an anchor primer having an attached capturable moiety may be purchased commercially such as, for example, a biotinylated anchor primer (Gibco Life Technologies, Grand Island, N.Y.) or an amine-modified anchor primer (Midland Certified Reagents, Midland, Tex.).

The capturable moiety may be attached directly to a nucleotide of the anchor primer. Alternatively, the capturable moiety may be attached indirectly to a nucleotide of the anchor primer via a spacer. In addition, the capturable moiety may be attached directly or indirectly to the substrate or solid-phase support. The spacer may encompass any suitable means that can be used to link a nucleotide of the anchor primer to the capturable moiety or any suitable means to link the capturable moiety to the solid-phase support. However, the spacer should not adversely affect the function of the anchor primer or the capturable moiety.

C. Cleavage of the cDNA Sample With Restriction Endonucleases

The cDNA sample is cleaved with a restriction endonuclease that recognizes at least a four nucleotide sequence not found within the sequence of the anchor primer. In general, suitable restriction endonucleases include AciI, AluI, BfaI, BstUI, Csp6I, DpnI, DpnIII, HaeIII, HhaI, HinP1I, HpaII, MaeII, MboI, MnlI, MseI, MspI, NlaIII, RsaI, Sau3AI, TaiI, TaqI, and Tsp509I. Such endonucleases typically cleave at multiple sites in most cDNAs. Typically, the restriction endonuclease is MspI. Alternatively, the restriction endonuclease can be TaqI, MaeII, HinP1I, Sau 3AI and NlaIII. These restriction endonucleases can be used to detect those rare mRNA species that are not cleaved by MspI.

In other embodiments, a suitable restriction endonuclease that recognizes at least a four nucleotide sequence not found within the sequence of the anchor primer can be selected from the group consisting of BglII, BmgI, BsaAI, BsaHI, BsaWI, BsbI, BsePI, BseSI, BsiI, BsiEI, BsiHKAI, BsiWI, BsmI, Bsp1286I, Bsp1407I, BspEI, BspGI, BspHI, BspLU11I, BspEI, BspGI, BspHI, BspLU11, BspMII, BsrBI, BsrFI, BsrGI, BspHI, BssHII, BssSI, BstBI, BstYI, BstZ17I, BtgI, BtrI, CfrI, Cfr10I, ClaI, DraI, DrdII, DsaI, EaeI, EagI, Ecl136II, Eco47III, EcoNI, EspI, Esp3I, FspI, GdiII, HaeI, HaeII, HgiAI, HgiEII, HgiJII, Hin4I, HincII, HindII, HindIII, KasI, KpnI, Ksp632I, LpnI, MfeI, MmeI, MscI, MslI, MspA1I, MstI, NaeI, NarI, NcoI, NdeI, NheI, Nli3877I, NotI, NruI, NspBII, OliI, PciI, PflMI, PmeI, Ppu10I, PpuMI, PspOMI, PsrI, PssI, PvuII, RleAI, RsrII, SaI, SauI, SbfI, SciI, SduI, SfcI, SfoI, SgfI, SgrAI, SmaI, SmaI, SnaI, SnaBI, SrfI, Sse232I, Sse8387I, Sse8647I, StyI, Tth111I, Tth111II, UbaKI, VspI, XbaI, XcmI, XhoI, XmaI. Alternatively, a suitable restriction endonuclease that recognizes at least a four nucleotide sequence not found within the sequence of the anchor primer can be selected from the group consisting of AscI, BaeI, FseI, NotI, PacI, PmeI, PpuMI, RsrII, SapI, SexAI, SfiI, SgfI, SgrAI, SrfI, Sse8387I and SwaI.

Conditions for digestion of the cDNA are well-known in the art and are described, for example, in J. Sambrook et al., “Molecular Cloning: A Laboratory Manual,” Vol. 1, Ch. 5, “Enzymes Used in Molecular Cloning.”

D. Capture of Double Stranded cDNA Restriction Fragments

In preferred embodiments of the invention, capture onto a magnetic bead substrate was used to improve the preparation of the population of cDNA molecules. Typically, the biotin moiety is conjugated to the 5′ terminus of the anchor primer and the first restricted cDNA is separated from the remainder of the cDNA by contacting the first restricted cDNA with a streptavidin-coated substrate, such as number of streptavidin coated magnetic beads.

E. Ligation of Adapter to Double Stranded cDNA Restriction Fragments

The captured double stranded cDNA fragments bound to the beads were then ligated to double stranded adapter polynucleotides that include a bacteriophage RNA polymerase promoter site to form adapted cDNA fragments. In one preferred embodiment, the CG overhang provided on the adapter polynucleotide allows for ligation to cDNA fragments cut with MspI having GC overhangs.

Several ligation reagent kits are commercially available, and the skilled artisan can alternatively assemble the components of the ligation reaction from individual sources. One suitable set of reagents is provided in the Rapid Ligation kit (Boehringer Mannheim). Preferably, the two individual oligonucleotides that form the double stranded adapter are synthetic and are annealed to each other before being added to the ligation reaction mixture.

F. Transcription of Synthetic RNA

In some preferred embodiments, synthetic RNA is prepared. This is performed by incubation of the linearized fragments with an RNA polymerase capable of initiating transcription from the bacteriophage-specific promoter. Typically, as discussed above, the promoter is a T3 promoter, and the polymerase is therefore T3 RNA polymerase. The polymerase is incubated with the linearized fragments and the four ribonucleoside triphosphates under conditions suitable for synthesis (Ambion, Austin, Tex.).

G. Synthesis of First-strand cDNA

First-strand cDNA is transcribed using Moloney murine leukemia virus (MMLV) reverse transcriptase (Life Technologies, Gaithersburg, Md.). With this reverse transcriptase annealing is performed at 42 degrees Celsius, and the transcription reaction at 42 degrees Celsius. The reaction uses a primer which is 15 to 30 nucleotides in length and complementary to the anchor sequence.

In another embodiment, the synthetic RNA is reverse transcribed using a thermostable reverse transcriptase and a primer as described below. A preferred reverse transcriptase is the avian recombinant reverse transcriptase, known as ThermoScript RT, available from Life Technologies (Gaithersburg, Md.). This step promotes high fidelity complementarity between the primer and the cRNA. The primer oligonucleotide used is at least 15 nucleotides in length, corresponding in sequence to the 3′-end of the bacteriophage-specific promoter.

Another suitable reverse transcriptase is the recombinant reverse transcriptase from Thermus thermophilus, known as rTth, available from Perkin-Elmer (Norwalk, Conn.).

H. First Polymerase Chain Reaction

The next step is the use of the cDNA product of reverse transcription, or, alternatively, the adapted cDNA fragment of step E, as a template for a polymerase chain reaction with a first set of primers as described below to produce sequence-specific PCR products.

In general, the cDNA is used as a template for a polymerase chain reaction with a first 3′ PCR primer and a first 5′ PCR primer to produce sequence-specific PCR products. The first 3′ PCR primer typically is 15 to 30 nucleotides in length, and is complementary to the anchor primer 3′ PCR primer correlate segment. The first 5′-PCR primers have a 3′ terminus consisting of -N_{X} where each “N_{X}” is one of the four deoxyribonucleotides A, C, G, or T, the primer being 15 to 30 nucleotides in length and complementary to the adapter polynucleotide sequence with the primer's complementarity extending into one nucleotide of the insert-specific nucleotides of the cDNA, wherein a different one of the first 5′ PCR primers is used in each of four different subpools.

Typically, PCR is performed using a PCR program of 15 seconds at 94 degrees Celsius for denaturation, 15 seconds at 50-65 degrees Celsius for annealing, and 30 seconds at 72 degrees Celsius for synthesis on a suitable thermocycler such as the PTC-200 (MJ Research) or the Perkin-Elmer 9600 (Perkin-Elmer Cetus, Norwalk, Conn.). The annealing temperature is optimized for the specific nucleotide sequence of the primer, using principles well known in the art. Performing the annealing step at a high temperature minimizes artifactual mispriming by the first 5′-PCR primer at its 3′-end and promotes high fidelity copying.

I. Second Polymerase Chain Reaction

The next step is the use of the products of the first PCR reaction as templates for a second polymerase chain reaction with a second set of primers as described below to produce a second set of sequence-specific PCR products.

In general, the product of first PCR reaction is used as a template for a polymerase chain reaction with the 3′ PCR primer as described above and a second 5′-PCR primer to produce sequence-specific PCR products. The second 5′ PCR primer is defined as having a 3′-terminus consisting of -N_{X}-N_{X+Y}, wherein N_{X} is the same as the NX used in the first polymerase chain reaction for that subpool, wherein X is an integer from 1 to 3, Y is an integer from 1 to 5, (X+Y) is an integer from 2 to 6, N is selected from group consisting of the four deoxyribonucleotides A, C, G, and T, wherein the second 5′ PCR primer is about 15 to about 30 nucleotides in length and wherein the second 5′ PCR-primer is complementary to a portion of the adapter polynucleotide with the complementarity extending X+Y nucleotides beyond the portion of the adapter polynucleotide into the specific sequence corresponding to the free end of the capturable cDNA, wherein a different one of the second 5′ PCR primers is used in the different 4^{X+Y} subpools of the second series of subpools.

In another embodiment, the 5′-PCR primer is selected from the group consisting of: (i) the first 5′ PCR primer which was used in the first PCR reaction for that subpool; (ii) the first 5′ PCR primer from which the first-strand cDNA was made for that subpool extended at its 3′-terminus by an additional residue -N; (iii) the first 5′ PCR primer used for that subpool extended at its 3′ terminus by two additional residues -N-N, (iv) the first 5′ PCR primer used for that subpool extended at its 3′ terminus by three additional residues -N-N-N; and (v) the first 5′ PCR primer used for that subpool extended at its 3′-terminus by four additional residues -N-N-N-N, wherein N can be any of A, C, G, or T.

Suitable 3′ PCR primers are selected from the group consisting of 5′-G-A-G-C-T-C-C-A-C-C-G-C-G-G-T-3′ (SEQ ID NO:5) and 5′-G-A-G-C-T-C-G-T-T-T-T-C-C-C-A-G-3′ (SEQ ID NO:6).

A suitable set of 5′-PCR primers is chosen to be capable of hybridizing to a corresponding specific double stranded adapter polynucleotide. For one preferred embodiment in which the adapter polynucleotide comprises SEQ ID NO:7 and SEQ ID NO:8, a set of 5′ PCR primers is appropriately chosen from the group consisting of the sequences:

C-T-C-G-A-G-C-T-C-G-A-C-G-G-T-A-T-C-G-G-N (SEQ ID NO:16);

C-C-T-C-G-A-G-G-T-C-G-A-C-G-G-T-A-T-C-G-G-N (SEQ ID NO: 17);

C-T-C-G-A-C-G-G-T-A-T-C-G-G-N-N (SEQ ID NO:30);

G-T-C-G-A-C-G-G-T-A-T-C-G-G-N-N (SEQ ID NO:41);

T-C-G-A-C-G-G-T-A-T-C-G-G-N-N-N (SEQ ID NO:31);

C-G-A-C-G-G-T-A-T-C-G-G-N-N-N-N (SEQ ID NO:18);

G-A-C-G-G-T-A-T-C-G-G-N-N-N-N-N (SEQ ID NO:32); and

A-C-G-G-T-A-T-C-G-G-N-N-N-N-N-N (SEQ ID NO:33).

Typically, PCR is performed using a PCR program of 15 seconds at 94 degrees Celsius denaturation, 15 seconds at 50-65 degrees Celsius for annealing, and 30 seconds at 72 degrees Celsius for synthesis using a suitable thermocycler such as the PTC-200 (MJ Research) or the Perkin-Elmer 9600 (Perkin-Elmer Cetus, Norwalk, Conn.). The annealing temperature is optimized for the specific nucleotide sequence of the primer, using principles well known in the art. The high temperature annealing step minimizes artifactual mispriming by the 5′-PCR primer at its 3′-end and promotes high fidelity copying.

In preferred embodiments, detection methods utilizing non-radioactive labels can be used. For non-radioactive detection methods, one of the primers for the second PCR reaction is preferably conjugated to a fluorescent label. In presenting preferred embodiments, the 3′-PCR primer is conjugated to a fluorescent label. A suitable fluorescent label is selected from the group consisting of

spiro(isobenzofuran-1(3H),9′-(9H)-xanthen)-3-one, 6-carboxylic acid, 3′,6′-dihydroxy-6-carboxyfluorescein (6-FAM, ABI);

spiro(isobenzofuran-1 (3H),9′-(9H)-xanthen)-3-one, 5-carboxylic acid, 3′,6′-dihydroxy-5-carboxyfluorescein (5-FAM, Molecular Probes);

spiro(isobenzofuran-1(3H), 9′-(9H)-xanthen)-3-one, 3′,6′-dihydroxy-fluorescein (FAM, Molecular Probes);

9-(2,5-dicarboxyphenyl)-3,6-bis(dimethylamino)-xanthylium (6-carboxytetramethylrhodamine (6-TAMRA), Molecular Probes);

3,6-diamino-9-(2-carboxyphenyl)-xanthylium (Rhodamine Green™, Molecular Probes); spiro[isobenzofuran-1(3H), 9′-xanthene]-6-carboxylic acid,5′-dichloro-3′,6′-dihydroxy-2′,7′-dimethoxy-3-oxo-(JOE, Molecular Probes);

1H,5H,11H,15H-xantheno[2,3,4-ij:5,6,7-i‘j’]diquinolizin-8-ium, -(2,4-disulfophenyl)-2,3,6,7,12,13,16,17-octahydro-, inner salt (Texas Red, Molecular Probes);

6-((4,4-difluoro-5,7-dimethyl-4-bora-3a,4a-diaza-s-indacene-3-propionyl) amino) hexanoic acid (BODIPY FL-X, Molecular Probes);

6-((4,4-difluoro-1,3-dimethyl-5-(4-methoxyphenyl)-4-bora-3a,4a-diaza-s-indacene-3-propionyl)amino)hexanoic acid (BODIPY TMR-X, Molecular Probes); 6-(((4-(4,4-difluoro-5-(2-thienyl)-4-bora-3a,4a-diaza-s-indacene-3-yl) phenoxy)acetyl) amino)-hexanoic acid (BODIPY TR-X, Molecular Probes);

4,4-difluoro-4-bora-3a,4a-diaza-s-indacene-3-pentanoic acid (BODIPY FL-C₅, Molecular Probes);

4,4-difluoro-5,7-dimethyl-4-bora-3a,4a-diaza-s-indacene-3-propanoic acid (BODIPY FL, Molecular Probes);

4,4-difluoro-5-phenyl-4-bora-3a,4a-diaza-s-indacene-3-propionic acid (BODIPY 581/591, Molecular Probes);

4,4-difluoro-5-(4-phenyl-1,3-butadienyl)-4-bora-3a,4a-diaza-s-indacene-3-propionic acid (BODIPY 564/570, Molecular Probes);

4,4-difluoro-5-styryl-4-bora-3a,4a-diaza-s-indacene-3-propionic acid;

6-(((4,4-difluoro-5-(2-thienyl)-4-bora-3a,4a-diaza-s-indacene-3-yl)styryloxy)acetyl) aminohexanoic acid (BODIPY 630/650, Molecular Probes);

6-(((4,4-difluoro-5-(2-pyrrolyl)-4-bora-3a,4a-diaza-s-indacene-3-yl) styryloxy)acetyl) aminohexanoic acid (BODIPY 650/665, Molecular Probes); and

9-(2,4(or 2,5)-dicarboxyphenyl)-3,6-bis(dimethylamino)-xanthylium, inner salt (TAMRA, Molecular Probes). Other suitable fluorescent labels, including 4, 7, 2′,4′,5′, 7′ hexachloro 6-carboxyfluorescein (“HEX,” ABI), 4, 7, 2′,7′ tetrachloro 6-carboxyfluorescein (“TET,” ABI) and “NED” (ABI) are known in the art.

A preferred fluorescent label is spiro(isobenzofuran-1(3H),9′-(9H)-xanthen)-3-one, 6-carboxylic acid, 3′,6′-dihydroxy-6-carboxyfluorescein (6-FAM).

In alternative embodiments, autoradiographic detection methods can be used. In one embodiment, the PCR is performed in the presence of ³⁵S-dATP. Alternatively, the PCR amplification can be carried out in the presence of a radionuclide labeled deoxyribonucleoside triphosphate, such as [³²P]dCTP or [³³P]dCTP. However, for autoradiographic detection it is generally preferred to use a ³⁵S-labeled deoxyribonucleoside triphosphate for maximum resolution.

In an alternative embodiment, the detection method employs oligonucleotides that are labeled with magnetic particles that are used and detected as described in U.S. Pat. No. 5,656,429, the teachings of which are incorporated by reference.

In one preferred embodiment, the three nucleotides at the 3′ end of the first or second 5′ PCR primer are joined by phosphorothioate linkages. See, Mullins, J. I., de Noronha, C. M. Amplimers with 3′-terminal phosphorothioate linkages resist degradation by vent polymerase and reduce Taq polymerase mispriming. PCR Methods Appl 1992 2(2):131-136; Ott, J. and Eckstein, F. Protection of oligonucleotide primers against degradation by DNA polymerase I. Biochemistry 1987 26(25):8237-8241; Uhlmann, E., Ryte, A., and Peyman, A. Studies on the mechanism of stabilization of partially phosphorothioated oligonucleotides against nucleolytic degradation. Antisense Nucleic Acid Drug Dev. 1997 7(4):345-350; Schreiber, G., Koch, E. M., and Neubert, W. J. Selective protection of in vitro synthesized cDNA against nucleases by incorporation of phosphorothioate-analogues. Nucleic Acids Res. 1985 13(21):7663-7672.

J. Resolution of the Products of the Second PCR

The detectable sequence-specific PCR products are then resolved by a separation method such as electrophoresis to display bands representing the 3′-ends of mRNA species present in the sample. Electrophoretic techniques for resolving the sequence-specific PCR products are well-understood in the art and need not be further recited here. In one preferred embodiment, the corresponding products are resolved in denaturing DNA sequencing gels and visualized by laser induced fluorescence. In another preferred embodiment, the corresponding sequence-specific PCR products are resolved using capillary electrophoresis and visualized by laser induced fluorescence.

Typically, laser induced fluorescence is used to detect the resolved cDNA species. However, other detection methods, such as phosphorimaging, autoradiography, magnetic detection, or mass spectrometry can be used.

According to the scheme, the population of cDNA molecules produced from each of the mRNA samples contain copies of the extreme 3′-ends from the most distal site for MspI to the beginning of the poly(A) tail of all poly(A)⁺ mRNA species in the starting RNA sample approximately according to the initial relative concentrations of the mRNA species. The lengths of the resulting PCR products are uniform for each species, allowing their later visualization as discrete bands on a gel or peaks in a TOGA™ profile, regardless of the tissue source of the mRNA.

Typically, the intensity of labeled PCR products displayed after electrophoresis is about proportional to the abundances of the niRNA species corresponding to the products in the original mixture.

Typically, the method further comprises a step of determining the relative abundance of each mRNA species in the original mixture from the intensity of the PCR product corresponding to that mRNA species after electrophoresis.

A further application of the method of the present invention is in obtaining the sequence of the 3′-ends of PCR product species that are displayed. Methods of determining the sequence of polynucleotides are well known to one skilled in the art. In one illustrative example, a method of obtaining the sequence comprises:

(1) eluting at least one PCR product corresponding to a mRNA species from an electropherogram in which bands representing the 3′-ends of mRNA species present in the sample are displayed;

(2) cloning the PCR product into a plasmid;

(3) producing DNA corresponding to the cloned DNA from the plasmid; and

(4) sequencing the cloned cDNA.

The PCR product that has been eluted can be amplified conveniently with the primers a previously used in the second PCR step. The PCR product can then be cloned into pCR II (Invitrogen, San Diego, Calif.) by TA cloning and ligation into the vector. Minipreps of the DNA can then be produced by standard techniques from subclones and a portion denatured and split into two aliquots for automated sequencing by the dideoxy chain termination method of Sanger. A commercially available sequencer can be used, such as an ABI sequencer, for automated sequencing. Generally the entire sequence of sequence-specific PCR products in the length range of 50-500 bp can be determined.

In one alternative embodiment, the sequence-specific PCR product was sequenced using a modification of a direct sequencing (“ds”) methodology (Innis et al., Proc. Nat'l. Acad. Sci., 85: 9436-9440 (1988)). PCR products were gel purified and PCR amplified again to incorporate sequencing primers at the 5′- and 3′-ends. The sequence addition was accomplished through 5′ and 3′ ds-primers containing M13 sequencing primer sequences (M13 forward and M13 reverse respectively) at their 5′ ends, followed by a linker sequence and a sequence complementary to the DST ends. Using the Clontech Taq Start antibody system, a master mix containing all components except the gel purified PCR product template was prepared, which contained sterile H₂O, 10× PCR II buffer, 10 mM dNTP, 25 mM MgCl₂, AmpliTaq/Antibody mix (1.1 μg/μl Taq antibody, 5 U/μl AmpliTaq), 100 ng/μl of 5′ ds-primer (5′-T-C-C-C-A-G-T-C-A-C-G-A-C-G-T-T-G-T-A-A-A-A-C-G-A-C-G-G-C-T-C-A-T-A-T-G-A-A-T-T-A-G-G-T-G-A-C-C-G-A-C-G-G-T-A-T-C-G-G-3′, SEQ ID NO:36), and 100 ng/μl of 3′ ds-primer (5′-C-A-G-C-G-G-A-T-A-A-C-A-A-T-T-T-C-A-C-A-C-A-G-G-G-A-G-C-T-C-C-A-C-C-G-C-G-G-T-G-G-C-G-G-C-C-3′, SEQ ID NO:37). After addition of the PCR product template, PCR was performed using the following program: 94° C., 4 minutes and 25 cycles of 94° C., 20 seconds; 65° C., 20 seconds; 72° C., 20 seconds; and 72° C. 4 minutes. The resulting amplified PCR product was gel purified.

The purified PCR product was sequenced using a standard protocol for ABI 3700 sequencing. Briefly, triplicate reactions in forward and reverse orientation (6 total reactions) were prepared, each reaction containing 5 μl of gel purified PCR product as template. In addition, the sequencing reactions contained 2 μl 2.5× sequencing buffer, 2 μl Big Dye Terminator mix, 1 μl of either the 5′ sequencing primer (5′-C-C-C-A-G-T-C-A-C-G-A-C-G-T-T-G-T-A-A-A-A-C-G-3′, SEQ ID NO:38), or the 3′ sequencing primer (5′-T-T-T-T-T-T-T-T-T-T-T-T-T-T-T-T-T-T-V-3′, where V=A, C, or G, SEQ ID NO:39) in a total volume of 10 11.

In an alternate embodiment, the 3′ sequencing primer had the sequence 5′-G-G-T-G-G-C-G-G-C-C-G-C-A-G-G-A-A-T-T-T-T-T-T-T-T-T-T-T-T-T-T-T-T-T-T-3′ (SEQ ID NO:40). PCR was performed using the following thermal cycling program: 96° C., 2 minutes and 29 cycles of 96° C., 15 seconds; 50° C., 15 seconds; 60° C., 4 minutes.

K. Data Processing System

The partial sequences obtained by any such suitable method, including those described above, can then be compared to sequences stored in general nucleotide sequence databases such as GenBank. Previously, one to a few sequences at a time could be compared to sequences in a general nucleotide sequence databases to recognize sequence identities and similarities using programs such as BLASTN and BLASTX. In contrast, the present invention provides a data processing system that compares multiple sequence-specific PCR products to multiple candidate corresponding nucleotide sequences stored in general nucleotide sequence databases and presents the results in a graphical user interface. It is an advantage of the present invention that such comparisons can be made, and candidate corresponding nucleotide sequences identified, before completely sequencing the sequence-specfic PCR products Because this method generates sequences from only the 3′-ends of mRNA species it is expected that open reading frames (ORFs) would be encountered only occasionally. For example, the 3′-untranslated regions of brain mRNA molecules are on average longer than 1300 nucleotides (J. G. Sutcliffe, 1988, supra). Potential ORFs can be examined for signature protein motifs.

In general, the obtained sequence of at least one second PCR product is determined was compared to one or more corresponding nucleotide sequences from a database of nucleotide sequences, the corresponding nucleotide sequences being delimited by the 3′-most recognition site for the restriction endonuclease that recognizes at least a four nucleotide sequence not found within the sequence of the anchor primer and the beginning of the poly(A) tail. In general, the expected length of the sequence-specific second PCR product is determined from the sum of the lengths of the corresponding nucleotide sequence determined from the database, the length of the 5′ PCR sequence hybridizable to the adapter sequence, and the length of the remaining anchor primer sequence, including the length of the 3′ PCR primer segment.

In a preferred embodiment, the present invention provides a data processing system for storing and displaying characteristics of polynucleotide fragments comprising, in combination, a graphical user interface for visually displaying characteristics of polynucleotide fragments, such as the sequence-specific PCR products, and at least one database for storing characteristics of polynucleotide fragments is stored on a computer-readable medium. Typically, the database is constructed comprising the data produced by the quantitation of the fragment length and relative abundance of sequence-specific PCR products, including the characteristics of fragment length, relative abundance and partial sequence for each sequence-specific PCR product. Typically, the database further comprises data concerning sequence relationships, gene mapping and cellular distributions.

An operating environment for the data processing system for preferred embodiments of the present invention includes a processing system with one or more high speed Central Processing Unit(s) (“CPU”) and a memory. The CPU may be electrical or biological. In accordance with the practices of persons skilled in the art of computer programming, the present invention is described below with reference to acts and symbolic representations of operations or instructions that are performed by the processing system, unless indicated otherwise. Such acts and operations or instructions are referred to as being “computer-executed” or “CPU executed.” It will be appreciated that acts and symbolically represented operations or instructions include the manipulation of electrical signals or biological signals by the CPU. An electrical system or biological system represents data bits which cause a resulting transformation or reduction of the electrical signals or biological signals, and the maintenance of data bits at memory locations in a memory system to thereby reconfigure or otherwise alter the CPU's operation, as well as other processing of signals. The memory locations where data bits are maintained are physical locations that have particular electrical, magnetic, optical, or organic properties corresponding to the data bits.

The data bits may also be maintained on a computer readable medium including magnetic disks, optical disks, organic memory, and any other volatile (e.g., Random Access Memory (“RAM”)) or non-volatile (e.g., Read-Only Memory (“ROM”)) mass storage system readable by the CPU. The computer readable medium includes cooperating or interconnected computer readable medium, which exist exclusively on the processing system or be distributed among multiple interconnected processing systems that may be local or remote to the processing system.

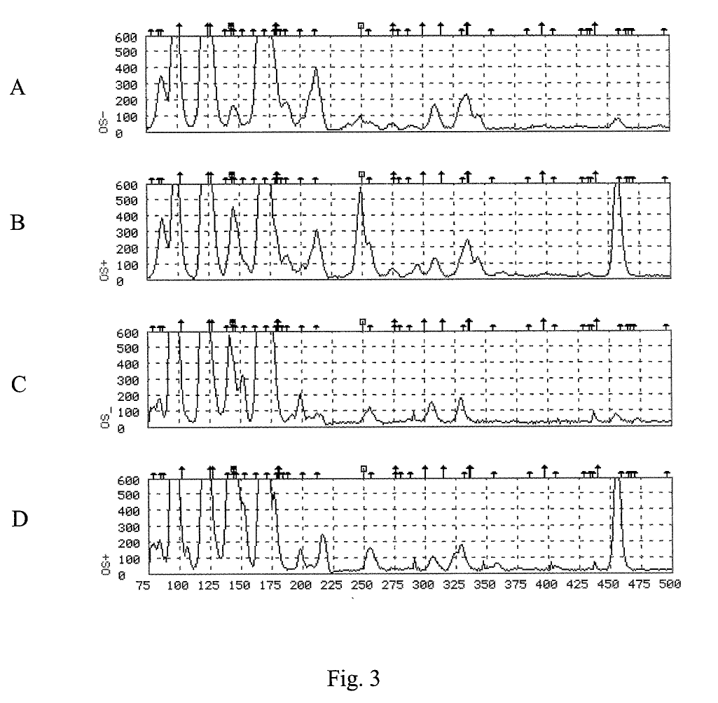

Preferably, comparing the length of the obtained sequence-specific PCR product to the determined expected length of the corresponding nucleotide sequence is presented in a graphical display in which at least two dimensions can be represented. Typically, the expected length of a sequence-specific PCR product based on a corresponding nucleotide sequence is indicated in the graphical display by the use of a graphical symbol or text character. Illustrative examples of graphical displays are provided in FIGS. 3-5. In these displays the expected length of a corresponding nucleotide sequence is indicated by symbols placed along the top edge of each panel. Typically, such a comparison results in the recognition of sequence identities and similarities between the sequence of the sequence-specific PCR product (and thereby, the sequence of 3′-ends of mRNA molecules present in a sample) and sequences selected from a general nucleotide sequence database.

In a preferred embodiment, a system is used for storing and displaying characteristics of polynucleotide fragments comprising the sequence-specific PCR products, comprising, in combination, a graphical user interface for visually displaying characteristics of polynucleotide fragment such as the sequence-specific PCR products, and a database for storing characteristics of polynucleotide fragments. Typically, the system comprises a central processing unit. Typically, the database is stored on a computer-readable medium. Typically, the database is constructed comprising the data produced by the quantitation of the fragment length and relative abundance of sequence-specific PCR products, including the characteristics of fragment length, relative abundance and partial sequence for each sequence-specific PCR product. Typically, the database further comprises data concerning sequence relationships, gene mapping and cellular distributions. Typically at least one database comprises corresponding nucleotide sequences determined from a database of nucleotide sequences, the corresponding nucleotide sequences being delimited by the 3′-most recognition site for the restriction endonuclease that recognizes at least a four nucleotide sequence not found within the sequence of the anchor primer and the beginning of the poly(A) tail. In general, an expected length of the sequence-specific second PCR product is determined from the sum of the lengths of the corresponding nucleotide sequence determined from the database, the length of the 5′ PCR sequence hybridizable to the adapter sequence, and the length of the remaining anchor primer sequence, including the length of the 3′ PCR primer segment of the anchor primer sequence.

The cDNA sequences obtained can then be used to design primer pairs for semiquantitative PCR to confirm tissue expression patterns. Selected products can also be used to isolate full-length cDNA clones for further analysis. Primer pairs can be used for SSCP-PCR (single strand conformation polymorphism-PCR) amplification of genomic DNA. For example, such amplification can be carried out from a panel of interspecific backcross mice to determine linkage of each PCR product to markers already linked. This can result in the mapping of new genes and can serve as a resource for identifying candidates for mapped mouse mutant loci and homologous human disease genes. SSCP-PCR uses synthetic oligonucleotide primers that amplify, via PCR, a small (100-200 bp) segment. (M. Orita et al., “Detection of Polymorphisms of Human DNA by Gel Electrophoresis as Single-Strand Conformation Polymorphisms,” Proc. Natl. Acad. Sci. USA 86: 2766-2770 (1989); M. Orita et al., “Rapid and Sensitive Detection of Point Mutations in DNA Polymorphisms Using the Polymerase Chain Reaction,” Genomics 5:874-879 (1989)).

The excised fragments of cDNA can be radiolabeled by techniques well-known in the art for use in probing a northern blot or for in situ hybridization to verify mRNA distribution and to learn the size and prevalence of the corresponding full-length mRNA. The probe can also be used to screen a cDNA library to isolate clones for more reliable and complete sequence determination. The labeled probes can also be used for any other purpose, such as studying in vitro expression.

In one embodiment, the method for the simultaneous sequence-specific identification of multiple mRNA molecules in a RNA population comprised the steps of:

preparing a population of capturable double-stranded cDNA molecules from a population of mRNA molecules having a 3′ poly (A) terminus by using a mixture of anchor primers, each anchor primer having a 5′ terminus and a 3′ terminus and including: (i) phasing residues located at the 3′ terminus of each of the anchor primers consisting of -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N; (ii) a tract of 8 to 40 T residues located towards the 5′-terminus relative to the phasing residues; (iii) a first stuffer segment consisting of 4 to 40 nucleotides; (iv) a segment complementary to a 3′ PCR primer consisting of about 12 to about 20 nucleotide residues located towards the 5′-terminus relative to the tract of T residues; (v) a second stuffer segment consisting of 4 to 40 nucleotides; (vi) at least one segment comprising a sequence recognized by a restriction endonuclease that recognizes at least six bases, the segment being located towards the 5′-terminus of the anchor primer relative to the 3′ PCR primer segment and (vii) a capturable moiety affixed to the anchor primer;

digesting the population of capturable double-stranded cDNA molecules with a restriction endonuclease that recognizes at least a four nucleotide sequence not found within the sequence of the anchor primer, thereby producing a population of capturable double stranded cDNA fragments, each capturable double stranded cDNA fragment having an anchor end that corresponds to the poly(A) segment of the original mRNA molecule and including at least a portion of a sequence corresponding to that of the anchor primer, and a free end opposite to the anchor end;

capturing the capturable moiety, thereby affixing the capturable double-stranded cDNA fragments to a substrate to form affixed double stranded cDNA fragments;

ligating a double stranded adapter polynucleotide to the free end of each affixed double stranded cDNA fragment to form a population of adapted cDNA molecules, the double stranded adapter polynucleotide including a segment corresponding to the sequence of a bacteriophage RNA polymerase promoter and a segment complementary to a 5′ PCR primer;

generating a first set of sequence-specific PCR products by dividing the population of adapted cDNA molecules into a first series of subpools as templates for a first polymerase chain reaction with a 3′ PCR-primer about 15 to 30 nucleotides in length that is complementary to at least a portion of the anchor primer sequence and a first 5′ PCR-primer about 15 to about 30 nucleotides in length and that is complementary to a portion of the adapter polynucleotide, with the complementarity extending one nucleotide beyond the portion of the adapter polynucleotide into the specific sequence corresponding to the free end of the capturable cDNA and including a 3′-terminus consisting of -N_{X}, wherein X is an integer from 1 to 5, and N is selected from group consisting of the four deoxyribonucleotides A, C, G, and T, and wherein a different one of the first 5′ PCR primers is used in each of 4^{X} different subpools;

generating a detectable second set of sequence-specific PCR products by further dividing the first set of sequence-specific PCR products in each of the first series of subpools into a second series of subpools and using the first set of sequence-specific PCR products as templates for a second polymerase chain reaction with a 3′ PCR primer of 15 to 30 nucleotides in length comprising a detectable moiety and a second 5′ PCR primer defined as having a 3′-terminus consisting of -N_{X}-N_{X+Y}, wherein NX is the same as the NX used in the first polymerase chain reaction for that subpool, wherein Y is an integer from 1 to 5, (X+Y) is an integer from 2 to 6, N is selected from group consisting of the four deoxyribonucleotides A, C, G, and T, wherein the second 5′ PCR primer is about 15 to about 30 nucleotides in length and wherein the second 5′ PCR-primer is complementary to a portion of the adapter polynucleotide with the complementarity extending X+Y nucleotides beyond the portion of the adapter polynucleotide into the specific sequence corresponding to the free end of the capturable cDNA, wherein a different one of the second 5′ PCR primers is used in the different 4^{X+Y} subpools of the second series of subpools;

resolving the second set of sequence-specific PCR products to generate a simultaneous display of sequence-specific PCR products representing the 3′-ends of mRNA molecules present in the mRNA population; and

characterizing each sequence-specific PCR product by a partial sequence and a length, thereby providing simultaneous sequence-specific identification of multiple mRNA molecules in a RNA population without making a cDNA library.

Typically, the step of preparing a population of double-stranded cDNA molecules comprises the steps of synthesizing a first cDNA strand and synthesizing a second cDNA strand.

Typically, the method further comprises the steps of transcribing to produce synthetic RNA molecules by incubating the capturable cDNA with a bacteriophage RNA polymerase capable of initiating transcription from the sequence corresponding to the sequence of a bacteriophage RNA polymerase promoter; and

generating first-strand cDNA by transcribing the cRNA using a reverse transcriptase and a RT primer being 15 to 30 nucleotides in length and comprising a segment capable of hybridizing to a portion of the anchor primer sequence.

The steps of the simplified TOGA™ method of the present invention have been automated to increase throughput and provide quality assurance for the larger number of PCR performed, the potentially different temperature optima for its 256 primers, and the provide the ability to conveniently apply the method to examine differences in mRNA expression patterns in many paradigms. See Sutcliffe, J. G., et al. (2000). Briefly, an Orca arm (Sagian, Indianapolis, Ind.) controlled from a computer terminal was used to transport disposable pipette tips and a bar-coded, 96-well reaction trays from a carousel to a Biomek liquid handling platform (Beckman), whose deck was maintained at 0° C. so that reactions remained inert during their assembly. The Orca arm collected bar-coded plates containing substrates, primers, and PCR reagents from a computer-controlled refrigerator, the bar codes were verified, and the Orca arm transfered the plates to the liquid handling platform, where the PCRs were assembled in batches on separate reaction trays, grouped according to their thermocycling programs. The Orca arm collected each reaction tray, placed it in a sealing device to insulate each well against evaporation and contamination, and the bar code was verified. The arm transported the tray to a thermocycler (PTC-200; MJ Research, Cambridge, Mass.) in which the PCR was executed under control of the computer. Upon completion of the reactions, the arm transferred the tray to the refrigerator.

In the embodiment described above, the arm serviced four thermocyclers, giving one station the capacity of analyzing 6,000 mRNA samples per year into 256 TOGA PCR product pools. Further refinements, equivalents and optimizations, such as substitution of a higher density format, e.g., 384 well plates for 96 well plates, can be made by the skilled artisan, and are considered within the scope of the present invention.

### EXAMPLE 1

### Original TOGA™ Method

The original TOGA™ (TOtal Gene expression Analysis) method, a method of simultaneous sequence-specific identification of mRNA molecules, was performed essentially as described in Sutcliffe, J. G., et al Proc Natl Acad Sci USA Feb. 29, 2000; 97(5): 1976-1981), international published application PCT/US99/23655, U.S. Pat. No. 5,459,037, U.S. Pat. No. 5,807,680, U.S. Pat. No. 6,030,784, U.S. Pat. No. 6,096,503, and U.S. Pat. No. 6,110,680, hereby incorporated herein by reference.

Briefly, prior to the application of the original TOGA™ technique, the isolated RNA was enriched to form a starting poly(A)-containing mRNA population by methods known in the art. The population of mRNA molecules used to generate the cDNA library may be isolated from any tissue or cell population, including cells grown in culture. Preferably, the mRNA is isolated from tissue or cells actively transcribing a potential gene of interest. Methods of extraction of RNA are well-known in the art and are described, for example, in Sambrook et al., Molecular Cloning: A Laboratory Manual (Cold Spring Harbor Press, Cold Spring Harbor, N.Y., 1989), vol. 1, ch.7, “Extraction, Purification, and Analysis of Messenger RNA from Eukaryotic Cells”, incorporated herein by this reference. Other isolation and extraction methods are also well-known.

Typically, RNA isolation is performed in the presence of chaotropic agents such as guanidinium chloride or guanidinium thiocyanate, although other detergents and extraction agents can alternatively be used. The mRNA (i.e. poly(A)⁺ enriched RNA) is typically isolated from the total extracted RNA by chromatography over oligo(dT)-cellulose or other chromatographic media that have the capacity to bind the polyadenylated 3′-end portion of mRNA molecules. Preferably, cDNA is prepared using poly(A)⁺ enriched mRNA. Also preferably, the poly(A)⁺ enriched mRNA is representative for all the expressed genes in the prepared sample. Typically, about 2 μg of mRNA is used to synthesize the cDNA. Alternatively, but less preferably, total RNA may be used to synthesize cDNA.

In a preferred embodiment, the TOGA method further comprised an additional PCR step performed using four 5′ PCR primers in four separate reactions and cDNA templates prepared from a population of antisense cRNAs. A final PCR step that used 256 5′ PCR primers in separate reactions produced PCR products that were cDNA fragments that corresponded to the 3′-region of the starting mRNA population. The produced PCR products were then identified by a) the initial 5′ sequence comprising the sequence remainder of the recognition site of the restriction endonuclease used to cut and isolate the 3′ region plus the sequence of the preferably four parsing bases immediately 3′ to the remainder of the recognition site, preferably the sequence of the entire fragment, and b) the length of the fragment. These two parameters, sequence and fragment length, were used to compare the obtained PCR products to a database of known polynucleotide sequences.

The method yields Digital Sequence Tags (DSTs), that is, polynucleotides that are expressed sequence tags of the 3′ end of mRNA molecules. DSTs that showed changes in relative levels as a result of experimental treatment were selected for further study. The intensities of the laser-induced fluorescence of the labeled PCR products were compared across samples.

In general, double-stranded cDNA is generated from poly(A)-enriched cytoplasmic RNA extracted from the tissue samples of interest using an equimolar mixture of all 48 5′-biotinylated anchor primers of a set to initiate reverse transcription.

One such suitable set is G-A-A-T-T-C-A-A-C-T-G-G-A-A-G-C-G-G-C-C-G-C-A-G-G-A-A-T-T-T-T-T-T-T-T-T-T-T-T-T-T-T-T-T-T-V-N-N (SEQ ID NO:4), where V is A, C or G and N is A, C, G or T. One member of this mixture of 48 anchor primers initiates synthesis at a fixed position at the 3′ end of all copies of each mRNA species in the sample, thereby defining a 3′ endpoint for each species, resulting in biotinylated double stranded cDNA.

Each biotinylated double stranded cDNA sample was cleaved with the restriction endonuclease MspI, which recognizes the sequence CCGG. The resulting fragments of cDNA corresponding to the 3′ region of the starting mRNA were then isolated by capture of the biotinylated cDNA fragments on a streptavidin-coated substrate. Suitable streptavidin-coated substrates include microtitre plates, PCR tubes, polystyrene beads, paramagnetic polymer beads and paramagnetic porous glass particles. A preferred streptavidin-coated substrate is a suspension of paramagnetic polymer beads (Dynal, Inc., Lake Success, N.Y.).

After washing the streptavidin-coated substrate and captured biotinylated cDNA fragments, the cDNA fragment product was released by digestion with NotI, which cleaves at an 8-nucleotide sequence within the anchor primers but rarely within the mRNA-derived portion of the cDNAs. The MspI-NotI fragments of cDNA corresponding to the 3′ region of the starting mRNA, which are of uniform length for each mRNA species, were directionally ligated into ClaI-NotI-cleaved plasmid pBC SK⁺ (Stratagene, La Jolla, Calif.) in an antisense orientation with respect to the vector's T3 promoter, and the product used to transform Escherichia coli SURE cells (Stratagene). The ligation regenerates the NotI site, but not the MspI site, leaving CGG as the first 3 bases of the 5′ end of all PCR products obtained. Each library contained in excess of 5×10⁵ recombinants to ensure a high likelihood that the 3′ ends of all mRNA species with concentrations of 0.001% or greater were multiply represented. Plasmid preps (Qiagen) were made from the cDNA library of each sample under study.

An aliquot of each library was digested with MspI, which effects linearization by cleavage at several sites within the parent vector while leaving the 3′ cDNA inserts and their flanking sequences, including the T3 promoter, intact. The product was incubated with T3 RNA polymerase (MEGAscript kit, Ambion) to generate antisense cRNA transcripts of the cloned inserts containing known vector sequences abutting the MspI and NotI sites from the original cDNAs.

At this stage, each of the cRNA preparations was processed in a three-step fashion. In step one, 250 ng of cRNA was converted to first-strand cDNA using the 5′ RT primer A-G-G-T-C-G-A-C-G-G-T-A-T-C-G-G, (SEQ ID NO:35). In step two, 400 pg of cDNA product was used as PCR template in four separate reactions with each of the four 5′ PCR primers of the form G-G-T-C-G-A-C-G-G-T-A-T-C-G-G-N (SEQ ID NO:34), each paired with a “universal” 3′ PCR primer, G-A-G-C-T-C-C-A-C-C-G-C-G-G-T (SEQ ID NO:5).

In step three, the product of each subpool was further divided into 64 subsubpools (2ng in 20 μl) for the second PCR reaction, with 100 ng each of the fluoresceinated 3′ PCR primer (SEQ ID NO:5) conjugated to 6-FAM and the appropriate 5′ PCR primer of the form C-G-A-C-G-G-T-A-T-C-G-G-N-N-N-N (SEQ ID NO:18), using a program that included an annealing step at a temperature X slightly above the Tₘ of each 5′ PCR primer to minimize artifactual mispriming and promote high fidelity copying. Each polymerase chain reaction step was performed in the presence of TaqStart antibody (Clonetech).

The products from the final polymerase chain reaction step for each of the tissue samples were resolved on a series of denaturing DNA sequencing gels using the automated ABI Prizm 377 sequencer. Data were collected using the GeneScan software package (ABI) and normalized for amplitude and migration. Complete execution of this series of reactions generated 64 product subpools for each of the four pools established by the 5′ PCR primers of the first PCR reaction, for a total of 256 product subpools for the entire 5′ PCR primer set of the second PCR reaction.

### EXAMPLE 2

Simplified TOGA Method with Intermediate Synthetic RNA Step

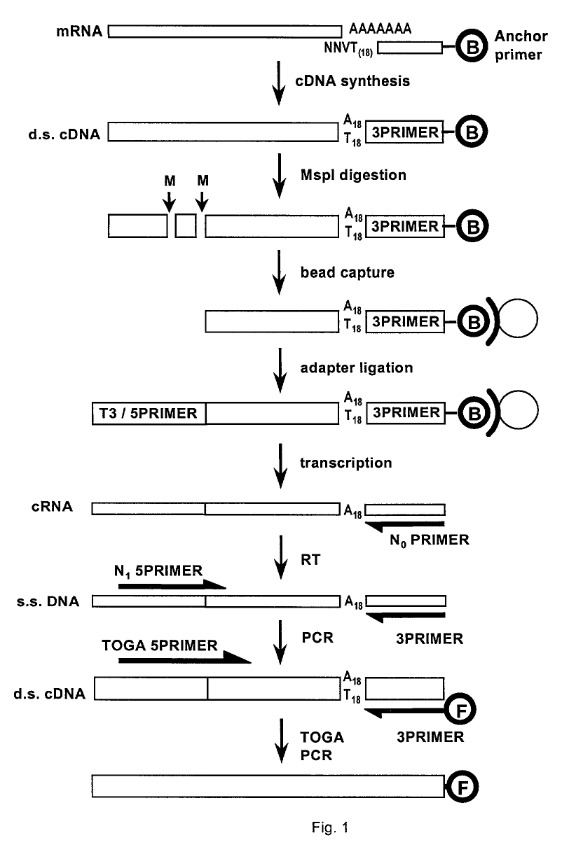

The steps of a preferred embodiment of the simplified method is illustrated diagramatically in FIG. 1. In this embodiment, the method comprises the steps of:

hybridizing an anchor primer having a capturable moiety to the poly (A) end of mRNA molecules;

producing a first strand of cDNA by reverse transcription;

synthesizing double stranded cDNA molecules using the first strand of cDNA as a template;

digesting the double stranded cDNA molecules using a restriction endonuclease to produce double stranded cDNA fragments;

capturing double stranded cDNA fragments, i.e. digestion fragments of the double stranded cDNA molecules, by means of the capturable moiety of the anchor primers;

ligating a double stranded DNA adapter polynucleotide to the end of the captured digestion fragments opposite the end affixed to the anchor primer to produce adapted cDNA molecules,

transcribing synthetic RNA from the adapted cDNA molecules,

producing a first strand of cDNA by reverse transcription of the synthetic RNA,

performing a first polymerase chain reaction step using a set of 5′ PCR N_{X} and 3′ PCR primers; and

performing a second polymerase chain reaction step using a set of 5′ PCR N_{X+Y} and 3′ PCR primers to produce multiple pools of sequence-specific PCR products.

Double-stranded cDNA was prepared from 2 μg of poly (A)-enriched cytoplasmic RNA extracted from the tissue or cell samples of interest by reverse transcription using an equimolar mixture of all 48 5′ biotinylated anchor primers of a set. The set of anchor primers (SEQ ID NO:1) in one preferred embodiment is described by Formula I, below.

```
                                                              Formula I
5′biotin-
ATGAATTCTCTAGAGATTGCTACCTCAGTCTGAGCTCCACCGCGGTAGTACTCACTGC(T)₁₈VNN-3′
    EcoRI XbaI 3′PCR primer oligo dT
```

In a more preferred embodiment, the anchor primers have the sequence 5′-A-T-G-A-A-G-C-A-G-T-T-T-T-T-T-T-T-T-T-T-T-T-T-T-T-T-T-V-N-N-3′ (SEQ ID NO: 2). In another more preferred embodiment, the anchor primers have the sequence 5′-G-A-A-T-T-C-A-A-C-T-G-G-C-T-G-C-A-G-T-T-T-T-T-T-T-T-T-T-T-T-T-T-T-T-T-T-V-N-N-3′ (SEQ ID NO: 3).

An aliquot (2 μg) of poly (A) enriched sample RNA was mixed with 0.5 μg of the anchor primer set mixture, diluted with diethyl pyrocarbonate (DEPC) treated water, held at 70 degrees Celsius for about 10 minutes, quenched in ice water and held on ice. To this was added the reverse transcriptase in the reaction mixture specified by the enzyme vendor: buffer, 0.1 M dithiothireitol (DTT), dNTPs, RNA Guard (Pharmacia-Upjohn) and 400 units of reverse transcriptase. A suitable reverse transcriptase is Superscript II RT (Life Technologies, MD). The reaction was allowed to proceed for the duration recommended by the vendor at the specified temperature, in this case, 1 hour at 42 degrees Celsius. The reaction was stopped by a brief centrifugation and storage on ice.

The second strand of cDNA was synthesized by adding to the cold first strand reaction mixture: buffer, dNTPs, RNAse H (Life Technologies), DEPC treated water and 25 units of DNA polymerase I (Life Technologies). The reaction was allowed to proceed for the duration recommended by the vendor at the specified temperature, in this case, incubation about 30 minutes incubation at 12 degrees Celsius; then about 1.5 hours at 22-25 degrees Celsius. The reaction was terminated by incubation at about 65 degrees Celsius for about 10 minutes, followed by a brief centrifugation and storage on ice. The double stranded cDNA was purified using phenol:chloroform extraction followed by a S-300 spin column.

Each double-stranded cDNA product was cleaved with the restriction endonuclease MspI, which recognizes the sequence CCGG. The 3′ fragments were isolated by capture on streptavidin coated beads (Dynabeads). The cDNA fragments bound to the beads were then ligated to 0.5 μg of double stranded adapters M1/M2 (respectively SEQ ID NO:7 and SEQ ID NO:8) that include the T3 bacteriophage RNA polymerase promoter site. The CG overhang provided on the M1 oligonucleotide allows for ligation to cDNA fragments cut with MspI having GC overhangs.

Several ligation reagent kits are commercially available, and the skilled artisan can alternatively assemble the components of the ligation reaction from individual sources. One suitable set of reagents is provided in the T4 DNA Ligation kit (Life Technologies). Preferably, the two individual oligonucleotides that form the double stranded adapter are synthetic and are annealed to each other before being added to the ligation reaction mixture. Typically, the oligonucleotide M2 was phosphorylated at the 5′ end using T4 polynucleotide kinase (BRL, 10 U/μl). The kinased oligonucleotide M2 was phenol:chloroform extracted, precipitated using 3 M NaOAc and ethanol, pelleted, washed with 70% ethanol and resuspended in water. An aliquot of M1 oligonucleotide is combined with the resuspended kinased M2 oligonucleotide and 5 M NaCl to a final concentration of 0.25 M NaCl. The combined M1 and M2 oligonucleotide mixture is incubated at 70 degrees Celsius for about 10 minutes, then placed in a 65 degrees Celsius water bath that is brought to room temperature slowly, permitting the formation of the double stranded M1/M2 adapter. The double stranded adapter was phenol:chloroform extracted, precipitated using 3 M NaOAc and ethanol, pelleted, washed with 70% ethanol, air dried at room temperature and resuspended in water.

The structure of a preferred double stranded adapter is given in Formula II, below.

```
                                                              FORMULA II
                                                                                                                    partial
                        KpnI T3 promoter TOGA primer
5′-ATGAATTCGGTACCAATTAACCCTCACTAAAGGGACAGCTTATCATCGCTCGAGCTCGACGGTAT-3′
3′-TACTTAAGCCATGGTTAATTGGGAGTGATTTCCCTGTCGAATAGTAGCGAGCTCGAGCTGCCATAGC-5′
```

The ligation of the double stranded adapter to the cDNA anchor fragments was accomplished using T4 ligase, ATP, and buffer. The ligated adapter-cDNA product was incubated with T3 RNA polymerase to generate sense synthetic RNA transcripts of the cDNA. Approximately 250 ng to 1 μg of the sense synthetic RNA was converted to first-strand cDNA by reverse transcription using the RT primer 5′-C-A-G-T-C-T-G-A-G-C-T-C-C-A-C-C-G-C-G-G-T-3′ (SEQ ID NO:15).

The cDNA product was then used as PCR templates with each of the four 5′ PCR N₁ primers of the sequence C-C-T-C-G-A-G-G-T-C-G-A-C-G-G-T-A-T-C-G-G-N (SEQ ID NO: 17) paired with the 3′ PCR primer (SEQ ID NO:5), using the program: 94 degrees Celsius for 15 seconds, 65 degrees Celsius for 15 seconds, and 72 degrees Celsius for 60 seconds, for 20 cycles. Alternatively, four 5′ PCR N₁ primers of the sequence C-T-C-G-A-G-C-T-C-G-A-C-G-G-T-A-T-C-G-G-N (SEQ ID NO:16) can be used.

Finally, the product of each subpool was divided further into 64 subsubpools (2 ng template in 20 μl reaction volume) for a second round of PCR reactions with 100 ng each of the fluorescently tagged 3′ PCR primer (SEQ ID NO:5) and the appropriate 5′ PCR N₄ primer of the sequence C-G-A-C-G-G-T-A-T-C-G-G-N-N-N-N (SEQ ID NO: 18) by using a program (94 degrees Celsius for 15 seconds, X degrees Celsius for 15 seconds, 72 degrees Celsius for 30 seconds, for 30 cycles) that included an annealing step at a temperature, X, slightly above the Tₘ of each N₄ primer to minimize artifactual mispriming and promote high-fidelity copying. The second round of PCR reactions were performed in the presence of TaqStart antibody.

Complete execution of the second round of PCR reactions generated 256 product pools for the entire 5′ PCR N₄ primer set for each of the cRNA samples. The products from each of the samples were resolved on a series of denaturing DNA sequencing gels by using the automated ABI Prism 377 DNA sequencer. Data were collected by using the GENESCAN software package provided by the manufacturer (Perkin-Elmer). The raw collected data were then filtered and smoothed to remove fluorescence noise. Lane migrations were calibrated to the migrations of intralane standards by a two-step interpolation. Peak amplitudes were normalized by using parameters determined on a panel-to-panel basis by interlane and intralane fluorescence-ratio statistics.

### EXAMPLE 3

### Simplified TOGA Method With Intermediate Synthetic RNA Step, Alternative Embodiment

In an alternative embodiment a different double stranded adapter polynucleotide was used. The steps of this variant of the method are substantially the same as discussed in Example 2 and illustrated diagramatically in FIG. 1. However, in this embodiment, a different double stranded adapter polynucleotide was used, comprising oligonucleotides O1 and O2 (SEQ ID NO:9 and SEQ ID NO:10), shown in Formula III, below. The positions of the KpnI recognition sequence, the T3 promoter site and TOGA primer site are underlined and labeled above the O2 sequence. The CG overhang provided on the O1 oligonucleotide allows for ligation to cDNA fragments that have been cut with MspI and thus have a GC overhang. The O1 oligonucleotide is phosphorylated, and may thus be used directly in the ligation step without treatment with kinase.

```
                                                              FORMULA III
                                                                                        partial
                KpnI T3 promoter TOGA primer
5′-AATTCGGTACCAATTAACCCTCACTAAAGGGACCTCGAGGTCGACGGTAT-3′
3′-TTAAGCCATGGTTAATTGGGAGTGATTTCCCTGGAGCTCCAGCTGCCATAGC-Phospho-5′
```

### EXAMPLE 4

### Simplified TOGA™ Method without an Intermediate Synthetic RNA Step

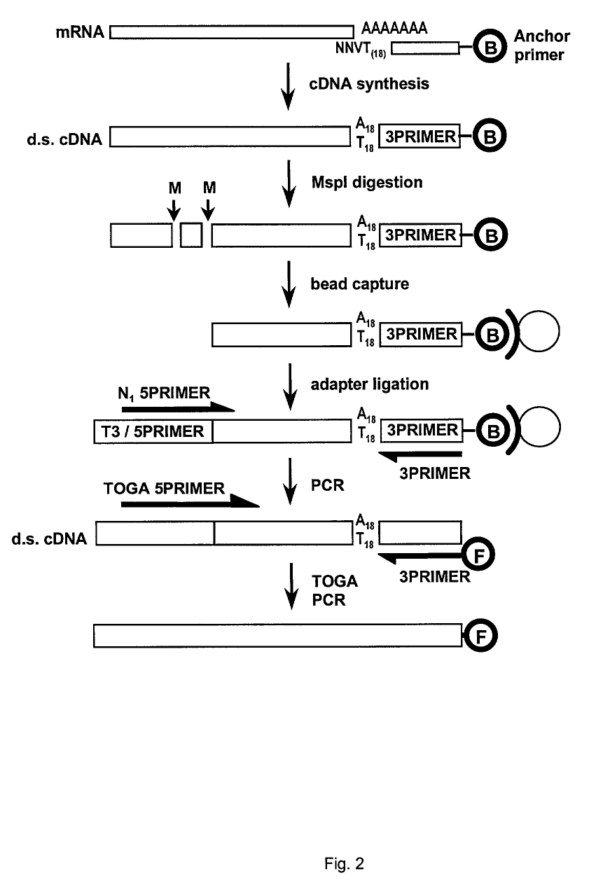

In another alternative embodiment, the synthetic RNA step can be omitted. The steps of this variant of the method are substantially the same as discussed in Example 3 with the omission of the steps of transcription of synthetic RNA. The steps of this alternative embodiment are illustrated diagramatically in FIG. 2.

Double-stranded cDNA was prepared from 2 μg of poly (A)-enriched cytoplasmic RNA extracted from the tissue or cell samples of interest by reverse transcription using an equimolar mixture of all forty eight 5′ biotinylated anchor primers of a set. In one preferred embodiment, a set of anchor primers disclosed in Example 2 (SEQ ID NO:1, NO:2 or NO:3) was used.

An aliquot (2 μg) of poly (A) enriched sample RNA was mixed with 0.5 μg of the anchor primer set mixture, diluted with diethyl pyrocarbonate (DEPC) treated water, held at 70 degrees Celsius for about 10 minutes, quenched in ice water and held on ice. To this was added the reverse transcriptase in the reaction mixture specified by the enzyme vendor: buffer, 0.1 M dithiothreitol (DTT), dNTPs, RNA Guard (Pharmacia-Upjohn) and 400 units of reverse transcriptase. A suitable reverse transcriptase is Superscript II RT (Life Technologies, MD). The reaction was allowed to proceed for the duration recommended by the vendor at the specified temperature, in this case, 1 hour at 42 degrees Celsius. The reaction was stopped by a brief centrifugation and storage on ice.

The second strand of cDNA was synthesized by adding to the cold first strand reaction mixture: buffer, dNTPs, RNAse H (Life Technologies), DEPC treated water and 25 units of DNA polymerase I (Life Technologies). The reaction was allowed to proceed for the duration recommended by the vendor at the specified temperature, in this case, incubation for about 30 minutes at about 12 degrees Celsius; then about 1.5 hours at 22-25 degrees Celsius. The reaction was stopped by incubation at about 65 degrees Celsius for about 10 minutes, followed by a brief centrifugation and storage on ice. The double stranded cDNA was purified using phenol:chloroform extraction followed by a S-300 spin column.

Each double-stranded cDNA product was cleaved with the restriction endonuclease MspI, which recognizes the sequence CCGG. The 3′ fragments were isolated by capture on streptavidin coated beads (Dynabeads). The cDNA fragments bound to the beads were then ligated to 0.5 μg of double stranded adapters O1/O2 (respectively SEQ ID NO:9 and SEQ ID NO:10) as described in Example 3.

The cDNA product was then used as PCR template with each of the four 5′ PCR N₁ primers of the sequence C-C-T-C-G-A-G-G-T-C-G-A-C-G-G-T-A-T-C-G-G-N (SEQ ID NO: 17) paired with the 3′ PCR primer (SEQ ID NO:5), using the program: 94 degrees Celsius for 15 seconds, 65 degrees Celsius for 15 seconds, and 72 degrees Celsius for 60 seconds, for 20 cycles. Alternatively, four 5′ PCR N₁ primers of the sequence C-T-C-G-A-G-C-T-C-G-A-C-G-G-T-A-T-C-G-G-N (SEQ ID NO:16) can be used.

Finally, the product of each subpool was divided further into 64 subsubpools (2 ng template in 20 μl reaction volume) for a second round of PCR reactions with 100 ng each of the fluorescently tagged 3′ PCR primer (SEQ ID NO:5) and the appropriate 5′ PCR N₄ primer of the sequence C-G-A-C-G-G-T-A-T-C-G-G-N-N-N-N (SEQ ID NO: 18) by using a program (94 degrees Celsius for 15 seconds, X degrees Celsius for 15 seconds, 72 degrees Celsius for 30 seconds, for 30 cycles) that included an annealing step at a temperature, X, slightly above the Tₘ of each N₄ primer to minimize artifactual mispriming and promote high-fidelity copying. The second round of PCR reactions were performed in the presence of TaqStart antibody.

Complete execution of the second round of PCR reactions generated 256 product pools for the entire 5′ PCR N₁N₂N₃N₄ primer set for each of the cRNA samples. The products from each of the samples were resolved on a series of denaturing DNA sequencing gels by using the automated ABI Prism 377 DNA sequencer. Data were collected by using the GENESCAN software package provided by the manufacturer (Perkin-Elmer). The raw collected data were then filtered and smoothed to remove fluorescence noise. Lane migrations were calibrated to the migrations of intralane standards by a two-step interpolation. Peak amplitudes were normalized by using parameters determined on a panel-to-panel basis by interlane and intralane fluorescence-ratio statistics.

### EXAMPLE 5

### Comparison of Results Obtained with Original And Simplified TOGA™ Methods.

TOGA display profiles from serum starved (FIGS. 3A and 3C) and serum replenished (FIGS. 3B and 3D) MG63 human osteosarcoma cells in original TOGA™ (FIGS. 3A and 3B) and simplified TOGA™ (FIGS. 3C and 3D). Serum starved MG63 cells were prepared by culturing the cells in medium lacking serum for 24 hours. Serum replenished MG63 cells were prepared by serum starving the cells for 24 hours followed by stimulation with medium containing 10% serum and anisomycin, a protein synthesis inhibitor, for 4 hours. Poly(A) enriched cytoplasmic RNA was isolated from serum starved and from serum replenished cells. Aliquots of the isolated mRNA were analyzed using both original TOGA™ and simplified TOGA™ techniques. Profiles were generated using 5′ PCR N₁N₂N₃N₄ primers having parsing bases CCCG.

Data are plotted as fluorescence intensity versus length of the PCR product (75 b.p.-500 b.p.) in base pairs. The predicted fragment lengths in original TOGA are offset from simplified TOGA by +2 bp.

### EXAMPLE 6

### Validation of Simplified TOGA™

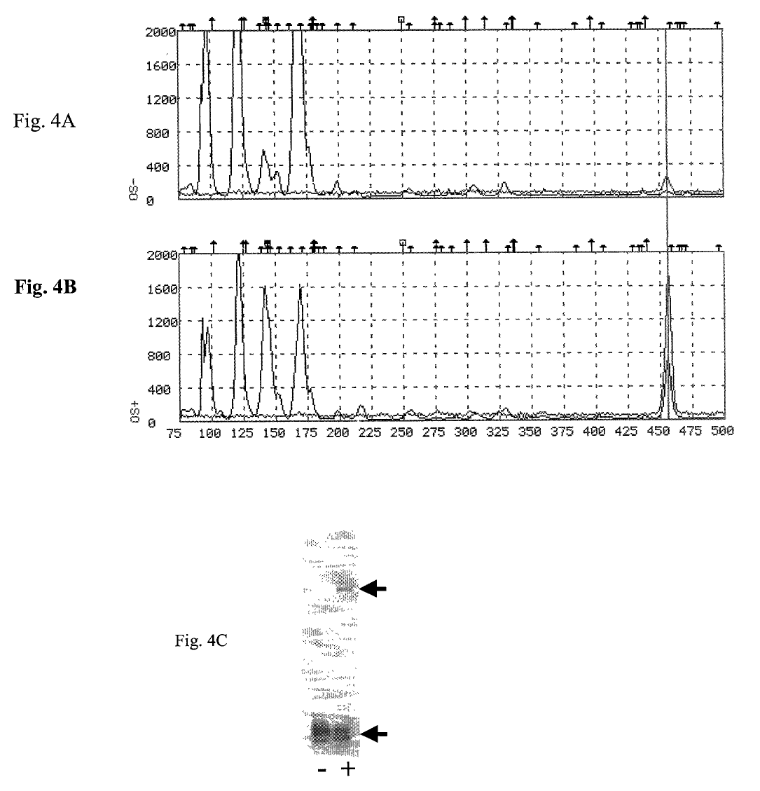

Simplified TOGA™ was performed as described in Example 2, above. Messenger RNA was prepared from serum starved and serum replenished MG63 cells as described in Example 5. TOGA display profiles were generated from serum starved (FIG. 4A) and serum replenished (FIG. 4B) MG63 human osteosarcoma cells. Profiles were generated using N₁N₂N₃N₄ primers having parsing bases CCCG (FIGS. 3A and 3B).

Data are plotted as fluorescence intensity versus length of the PCR product (75 b.p.-500 b.p.) in base pairs. The vertical guideline highlights a peak corresponding to a virtual DST (i.e., having the expected digital address, length and partial sequence) of NF-κB. The traces containing the highlighted peaks are shown overlaid with traces generated from 14 nucleotide extended primer specific for NF-κB (5′-G-A-T-C-G-A-A-T-C-C-G-G-C-C-C-G-C-C-T-G-A-A-T-C-A-T-T-C-T-C-3′, SEQ ID NO:25).

FIG. 4C shows a Northern blot prepared from RNA samples of serum starved (“-”) and serum replenished (“+”) MG63 cells. The blot was hybridized with human probes for NF-κB (identified in the TOGA panel CCCG) and ribosomal protein S20. The upper band, indicated by an arrow, corresponding to NF-κB was enriched in serum replenished cells confirming the finding in FIG. 4B. The lower band, indicated by an arrow, represents the mRNA for ribosomal protein S20, which was used as the normalization standard.

### EXAMPLE 6

### Reproducibilty of Simplified TOGA Methods

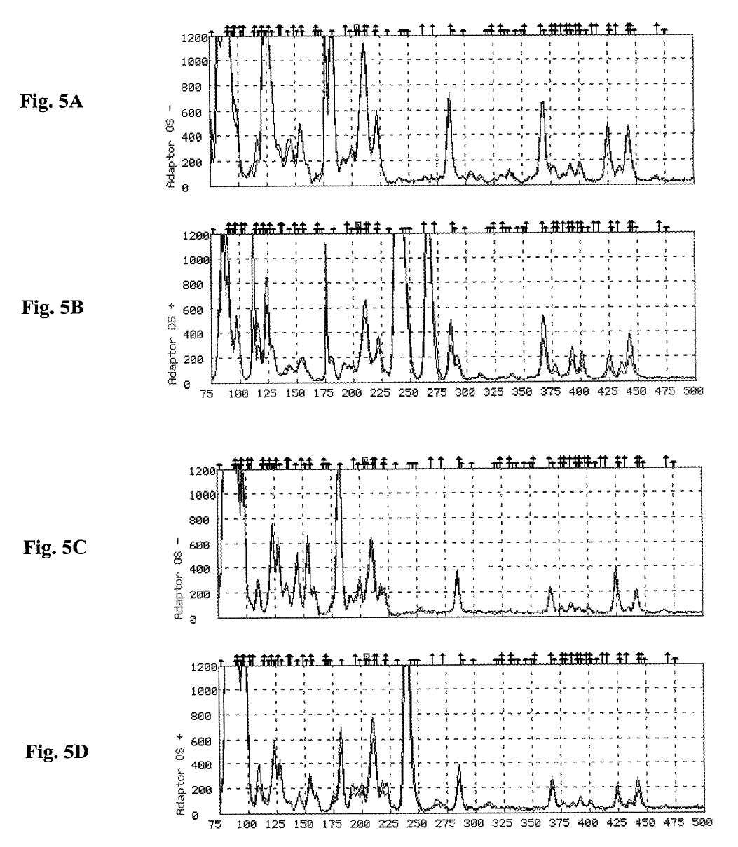

Serum starved and serum replenished MG63 cells were prepared as described in Example 5. TOGA display profiles were generated from serum starved (FIG. 5A) and serum replenished (FIG. 5B) MG63 human osteosarcoma cells. Profiles were generated using N₁N₂N₃N₄ primers having parsing bases ACTC. Simplified TOGA™ was performed in duplicate according to the method of Example 3 (FIGS. 5A and 5B) or according to the method of Example 4 (FIGS. 5C and 5D). Data from duplicate runs are shown overlaid in each panel, showing a high degree of reproducibility in peak position and amplitude.

TABLE 1

| | Original | Simplified | Simplified |
|---|---|---|---|
| | TOGA™ | TOGA™ | TOGA™ |
| Method: | Example 1 | Example 3 | Example 4 |
| Sample: | OS + RNA | OS + RNA | OS + RNA |
| Comparison: | Sample 1 vs 2 | Sample 1 vs 2 | Sample 1 vs 2 |
| Parsing Bases of | Correlation | Correlation | Correlation |
| 5′PCR Primer | Coefficient | Coefficient | Coefficient |
| ACAT | 9.892E-01 | 9.933E-01 | 9.974E-01 |
| ACTA | 9.724E-01 | 9.916E-01 | 9.942E-01 |
| ACTC | 9.593E-01 | 9.482E-01 | 9.794E-01 |
| ACTT | 9.656E-01 | 9.922E-01 | 9.377E-01 |
| CACG | 9.608E-01 | 9.949E-01 | 9.908E-01 |
| CGAT | 9.323E-01 | 9.503E-01 | 9.703E-01 |
| CTGA | 9.841E-01 | 9.729E-01 | 9.771E-01 |
| CTTC | 9.311E-01 | 9.967E-01 | 9.857E-01 |
| GGCA | 9.415E-01 | 9.978E-01 | 9.903E-01 |
| GGGT | 9.747E-01 | 9.974E-01 | 9.952E-01 |
| GTAA | 9.688E-01 | 9.969E-01 | 9.956E-01 |
| GTGA | 9.152E-01 | 9.970E-01 | 9.834E-01 |
| TACA | 9.734E-01 | 9.987E-01 | 9.950E-01 |
| TGAC | 9.875E-01 | 9.989E-01 | 9.936E-01 |
| TTAC | 7.680E-01 | 9.865E-01 | 9.580E-01 |
| TTGT | 9.493E-01 | 9.978E-01 | 9.921E-01 |
| Mean (N = 16) | 9.483E-01 | 9.882E-01 | 9.835E-01 |
| Variance | 5.266E-02 | 1.651E-02 | 1.628E-02 |
| 5th Percentile | 9.152E-01 | 9.503E-01 | 9.580E-01 |
| 25th Percentile | 9.415E-01 | 9.916E-01 | 9.794E-01 |
| 75th Percentile | 9.734E-01 | 9.974E-01 | 9.942E-01 |
| 95th Percentile | 9.875E-01 | 9.987E-01 | 9.956E-01 |

The reproducibility of these two embodiments of the simplified TOGA™ method and that of original TOGA™ method was examined in more detail by calculating the correlation coefficents of the TOGA™ profiles of PCR products obtained from duplicate mRNA samples for each of 16 N₁N₂N₃N₄ primers having parsing bases as listed in Table 1, above. The reproducibility, measured as the average correlation coefficient, of the original TOGA™ method is quite high, 0.948±0.053 (mean±variance). However, the reproducibility, measured as the average correlation coefficient, of the two embodiments of the simpified TOGA™ methods of the present invention is even higher. The simplified TOGA™ method of Example 3 produced an average correlation coefficient of 0.988±0.017 (mean±variance). The simplified TOGA™ method of Example 4 produced an average correlation coefficient of 0.984±0.016 (mean±variance). Note that the variance of the correlation coefficients is comparable for the variants of the simplified TOGA™ method, and about three-fold lower than that of the original TOGA™ method.

All of the references cited herein, including patents, published patent applications and publications, are hereby incorporated by reference. While the invention has been described with an emphasis upon preferred aspects of the invention, it will be readily apparent to those of ordinary skill in the art that variations of the preferred embodiments can be used and that it is intended that the invention can be practiced otherwise than is specifically described herein. Accordingly, the present invention includes all modifications encompassed within the spirit and scope of the invention as defined by the following claims.

We claim:

1. A simplified method for simultaneous sequence-specific identification of multiple mRNA molecules in a RNA population comprising the steps of:
preparing a population of capturable double-stranded cDNA molecules from a population of mRNA molecules having a 3′ poly (A) terminus by using a mixture of anchor primers, each anchor primer having a 5′ terminus and a 3′ terminus and including: (i) phasing residues located at the 3′ terminus of each of the anchor primers selected from the group consisting of -V, -V-N, and -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N where the anchor primer phasing residues in the mixture are defined by one of -V, -V-N, or -V-N-N; (ii) a tract of 8 to 40 T residues located towards the 5′-terminus relative to the phasing residues; (iii) a first stuffer segment consisting of 4 to 40 nucleotides; (iv) a segment complementary to a 3′ PCR primer consisting of about 12 to about 20 nucleotide residues located towards the 5′-terminus relative to the tract of T residues; (v) a second stuffer segment consisting of 4 to 40 nucleotides and (vi) a capturable moiety affixed to the anchor primer;
digesting the population of capturable double-stranded cDNA molecules with a restriction endonuclease that recognizes at least a four nucleotide sequence not found within the sequence of the anchor primer, thereby producing a population of capturable double stranded cDNA fragments, each capturable double stranded cDNA fragment having an anchor end that corresponds to the poly(A) segment of the original mRNA molecule and including at least a portion of a sequence corresponding to that of the anchor primer, and a free end opposite to the anchor end;
capturing the capturable moiety, thereby affixing the capturable double-stranded cDNA fragments to a substrate to form affixed double stranded cDNA fragments;
ligating a double stranded adapter polynucleotide to the free end of each affixed double stranded cDNA fragment to form a population of adapted cDNA molecules, the double stranded adapter polynucleotide including a segment corresponding to the sequence of a bacteriophage RNA polymerase promoter and a segment complementary to a 5′ PCR primer;
generating a first set of sequence-specific PCR products by dividing the population of adapted cDNA molecules into a first series of subpools as templates for a first polymerase chain reaction with a 3′ PCR-primer about 15 to 30 nucleotides in length that is complementary to at least a portion of the anchor primer sequence and a first 5′ PCR-primer about 15 to about 30 nucleotides in length and that is complementary to a portion of the adapter polynucleotide, with the complementarity extending one nucleotide beyond the portion of the adapter polynucleotide into the specific sequence corresponding to the free end of the capturable cDNA and including a 3′-terminus consisting of -N_{X}, wherein X is an integer from 1 to 5, and N is selected from group consisting of the four deoxyribonucleotides A, C, G, and T, and wherein a different one of the first 5′ PCR primers is used in each of 4^{X} different subpools;
generating a detectable second set of sequence-specific PCR products by further dividing the first set of sequence-specific PCR products in each of the first series of subpools into a second series of subpools and using the first set of sequence-specific PCR products as templates for a second polymerase chain reaction with a 3′ PCR primer of 15 to 30 nucleotides in length comprising a detectable moiety and a second 5′ PCR primer defined as having a 3′-terminus consisting of -N_{X}-N_{X+Y}, wherein N_{X} is the same as the N_{X} used in the first polymerase chain reaction for that subpool, wherein Y is an integer from 1 to 5, (X+Y) is an integer from 2 to 6, N is selected from group consisting of the four deoxyribonucleotides A, C, G, and T, wherein the second 5′ PCR primer is about 15 to about 30 nucleotides in length and wherein the second 5′ PCR-primer is complementary to a portion of the adapter polynucleotide with the complementarity extending X+Y nucleotides beyond the portion of the adapter polynucleotide into the specific sequence corresponding to the free end of the capturable cDNA, wherein a different one of the second 5′ PCR primers is used in the different 4^{X+Y} subpools of the second series of subpools;
resolving the second set of sequence-specific PCR products to generate a simultaneous display of sequence-specific PCR products representing the 3′-ends of mRNA molecules present in the mRNA population; and
characterizing each sequence-specific PCR product by a partial sequence and a length, thereby providing simultaneous sequence-specific identification of multiple mRNA molecules in a RNA population without making a cDNA library.

2. The method of claim 1, wherein the step of preparing a population of double-stranded cDNA molecules comprises the steps of synthesizing a first cDNA strand and synthesizing a second cDNA strand.

3. The method of claim 1, further comprising the steps of
transcribing to produce synthetic RNA molecules by incubating the capturable cDNA with a bacteriophage RNA polymerase capable of initiating transcription from the sequence corresponding to the sequence of a bacteriophage RNA polymerase promoter; and
generating first-strand cDNA by transcribing the cRNA using a reverse transcriptase and a RT primer being 15 to 30 nucleotides in length and comprising a segment capable of hybridizing to a portion of the anchor primer sequence.

4. The method of claim 1, wherein the anchor primer further comprises at least one segment comprising a sequence recognized by a restriction endonuclease that recognizes at least six bases, the segment being located towards the 5′-terminus of the anchor primer relative to the 3′ PCR primer segment.

5. The method of claim 1 wherein the anchor primer phasing residues are -V-N-N.

6. The method of claim 1 wherein X=1 and Y=3.

7. The method of claim 1 wherein the tract of T residues comprises 18 T residues.

8. The method of claim 1 wherein the anchor primer is chosen from the group consisting of primers having the sequence 5′-A-T-G-A-A-T-T-C-T-C-T-A-G-A-G-A-T-T-G-C-T-A-C-C-T-C-A-G-T-C-T-G-A-G-C-T-C-C-A-C-C-G-C-G-G-T-A-G-T-A-C-T-C-A-C-T-G-C-T-T-T-T-T-T-T-T-T-T-T-T-T-T-T-T-T-T-V-N-N-3′ (SEQ ID NO: 1), 5′-A-T-G-A-A-T-T-C-T-C-T-A-G-A-G-T-C-T-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 2) and 5′-G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 3), wherein the 5′ terminal base (base 1) is a biotinylated adenylate residue, each V can represent A, C or G, and each N can represent A, C, G, or T.

9. The method of claim 1 wherein the bacteriophage RNA polymerase promoter is selected from the group consisting of T3 promoter, T7 promoter and SP6 promoter.

10. The method of claim 9 wherein the bacteriophage RNA polymerase promoter is a T3 promoter.

11. The method of claim 1 wherein one strand of the double stranded adapter polynucleotide comprises the sequence 5′-A-T-G-A-A-T-T-C-G-G-T-A-C-C-A-A-T-T-A-A-C-C-C-T-C-A-C-T-A-A-A-G-G-G-A-C-A-G-C-T-T-A-T-C-A-T-C-G-C-T-C-G-A-G-C-T-C-G-A-C-G-G-T-A-T-3′ (SEQ ID NO:7).

12. The method of claim 1 wherein one strand of the double stranded adapter polynucleotide comprises the sequence 5′-C-G-A-T-A-C-C-G-T-C-G-A-G-C-T-C-G-A-G-C-G-A-T-G-A-T-A-A-G-C-T-G-T-C-C-C-T-T-T-A-G-T-G-A-G-G-G-T-T-A-A-T-T-G-G-T-A-C-C-G-A-A-T-T-C-A-T-3′ (SEQ ID NO:8).

13. The method of claim 1 wherein one strand of the double stranded adapter polynucleotide comprises the sequence 5′-Phospho-C-G-A-T-A-C-C-G-T-C-G-A-C-C-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:9).

14. The method of claim 1 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-G-G-T-C-G-A-C-G-G-T-A-T-3′ (SEQ ID NO: 10).

15. The method of claim 1 wherein one strand of the double stranded adapter polynucleotide comprises the sequence 5′-Phospho-G-A-T-C-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:11).

16. The method of claim 1 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-3′ (SEQ ID NO:12).

17. The method of claim 1 wherein one strand of the double stranded adapter polynucleotide comprises the sequence 5′-Phospho-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO: 13).

18. The method of claim 1 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-C-A-T-G-3′ (SEQ ID NO:14).

19. The method of claim 1 wherein the 3′ PCR primer comprises a sequence chosen from the group consisting of 5′-G-A-G-C-T-C-C-A-C-C-G-C-G-G-T-3′ (SEQ ID NO:5)) and 5′-G-A-G-C-T-C-G-T-T-T-T-C-C-C-A-G-3′ (SEQ ID NO:6).

20. The method of claim 4 wherein the RT primer comprises a sequence chosen from the group consisting of 5′-C-A-G-T-C-T-G-A-G-C-T-C-C-A-C-C-G-C-G-G-T-3′ (SEQ ID NO:15) and 5′-G-A-G-C-T-C-C-A-C-C-G-C-G-G-T-3′ (SEQ ID NO:5).

21. The method of claim 1 wherein the first 5′ PCR primer comprises a sequence chosen from the group consisting of 5′-C-T-C-G-A-G-C-T-C-G-A-C-G-G-T-A-T-C-G-G-N-3′ (SEQ ID NO:16), 5′-C-C-T-C-G-A-G-G-T-C-G-A-C-G-G-T-A-T-C-G-G-N-3′ (SEQ ID NO:17), 5′ A-G-C-T-C-T-G-T-G-G-T-G-A-G-G-A-T-C-N-3′ (SEQ ID NO:19), 5′-A-G-C-T-C-T-G-T-G-G-T-G-A-G-C-A-T-G-N-3′ (SEQ ID NO:21) and 5′-C-C-T-C-G-A-G-G-T-C-G-A-C-G-G-T-A-T-C-G-A-N -3′ (SEQ ID NO:23).

22. The method of claim 1 wherein the second 5′ PCR primer comprises a sequence chosen from the group consisting of 5′-C-G-A-C-G-G-T-A-T-C-G-G-N-N-N-N-3′ (SEQ ID
NO: 18), 5′-C-T-C-T-G-T-G-G-T-G-A-G-G-A-T-C-N-N-N-N-3′ (SEQ ID NO:20), 5′-C-T-C-T-G-T-G-G-T-G-A-G-C-A-T-G-N-N-N-N-3′ (SEQ ID NO:22), and 5′-T-C-G-A-G-G-T-C-G-A-C-G-G-T-A-T-C-G-A-N-N-N-N-3′ (SEQ ID NO:24).

23. The method of claim 1 wherein the three nucleotides at the 3′ end of the first 5′ PCR primer are joined by phosophodiesterase-resistant linkages.

24. The method of claim 1 wherein the restriction endonuclease recognizing at least a four nucleotide sequence not found within the sequence of the anchor primer is selected from the group consisting of AciI, AluI, BfaI, BstUI, Csp6I, DpnI, DpnII, HaeIII, HhaI, HinP1I, HpaII, MaeII, MboI, MnlI, MseI, MspI, NlaIII, RsaI, Sau3AI, TaiI, TaI, and Tsp509I.

25. The method of claim 1 wherein the restriction endonuclease recognizing a four-nucleotide sequence is Msp I

26. The method of claim 1 wherein the restriction endonuclease recognizing at least a four nucleotide sequence not found within the sequence of the anchor primer is selected from the group consisting of BglII, BmgI, BsaAI, BsaHI, BsaWI, BsbI, BsePI, BseSI, BsiI, BsiEI, BsiHKAI, BsiWI, BsmI, Bsp1286I, Bsp1407I, BspEI, BspGI, BspHI, BspLU11I, BspEI, BspGI, BspHI, BspLU11I, BspMII, BsrBI, BsrFI, BsrGI, BssHII, BssHII, BssSI, BstBI, BstYI, BstZ17I, BtgI, BtrI, CfrI, Cfr10I, ClaI, DraI, DrdII, DsaI, EaeI, EagI, Ecl136II, Eco47III, EcoNI, EspI, Esp3I, FspI, GdiII, HaeI, HaeII, HgiAI, HgiEII, HgiJII, Hin4I, HincII, HindII, HindIII, KasI, KpnI, Ksp632I, LpnI, MfeI, MmeI, MscI, MslI, MspA1I, MstI, NaeI, NarI, NcoI, NdeI, NheI, Nli3877I, NotI, NruI, NspBII, OliI, PciI, PflMI, PmeI, Ppu10I, PpuMI, PspOMI, PsrI, PssI, PvuII, RleAI, RsrII, SapI, SauI, SbfI, SciI, SduI, SfcI, SfoI, SgfI, SgrAI, SmaI, SmlI, SnaI, SnaBI, SrfI, Sse232I, Sse8387I, Sse8647I, StyI, Tth111I, Tth111II, UbaKI, VspI, XbaI, XcmI, XhoI, XmaI.

27. The method of claim 1 wherein the restriction endonuclease recognizing at least a four nucleotide sequence not found within the sequence of the anchor prime is selected from the group consisting of AscI, BaeI, FseI, NotI, PacI, PmeI, PpuMI, RsrII, SaI, SexAI, SfiI, SgfI, SgrAI, SrfI, Sse8387I and SwaI.

28. The method of claim 4 wherein the restriction endonuclease that recognizes at least six bases is selected from the group consisting of AscI, BaeI, FseI, NotI, PacI, PmeI, PpuMI, RsrII, SapI, SexAI, SfiI, SgfI, SgrAI, SrfI, Sse8387I and SwaI.

29. The method of claim 4 wherein the restriction endonuclease that recognizes at least six bases is selected from the group consisting of EcoRl and XbaI.

30. The method of claim 1, wherein the capturable moiety of the anchor primer of the double stranded DNA molecule is affixed to a substrate comprising a coating selected from the group consisting of streptavidin, avidin, neutravidin, N-oxysuccinimide ester, dimethyladipimidate-2-HCl, dimethylpimelimidate-HCl, dimethylsuberimidate-2HCl, dimethyl 3,3′-dithiobisproprionimidate-2HCl, disuccinimidyl glutarate, disuccinimidyl suberate, bis(sulfosuccinimidyl)suberate, dithiobis(succinimidyl proprionate), dithiobis(sulfosuccinimidyl proprionate), ethylene glycobis(succinimidylsuccinate), ethylene glycobis(sulfosuccinimidylsuccinate), disuccinimidyl tartarate, disulfosuccinimidyl tartarate, bis[2-succinimidyloxycarbonyloxy)ethyl]sulfone, bis[2-(sulfosuccinimidyloxycarbonyloxy) ethyl]sulfone, and N-hydroxysuccinimidyl 2,3-dibromopropionate, succinimidyl 4-(N-maleimidomethyl) cyclohexane-1-carboxylate, sulfosuccinimidyl 4-(N-maleimidomethyl) cyclohexane-1-carboxylate, m-maleimidobenzoyl-N-hydroxysuccinimide ester, m-maleimidobenzoyl-N-hydroxysulfosuccinimide ester, succinimidyl 4-(p-maleimidophenyl)-butyrate, sulfosuccinimidyl 4-(p-maleimidophenyl)-butyrate, bismaleimidohexane, N-(γ-maleimidobutyryloxy)succinimide ester, N-(γ-maleimidobutyryloxy)sulfosuccinimide ester, N-succinimidyl(4-iodoacetyl)aminobenzoate, sulfosuccinimidyl(4-iodoacetyl)aminobenzoate, 1,4-Di-[3′-2′-pyridyldithio(propionamido)butane], 4-succinimidyloxycarbonyl-α-(2-pyridyldithio)toluene, sulfosuccinimidyl-6-[α-methyl-α-(2-pyridyldithio)-toluamido]hexane, N-succinimidyl-3-(2-pyridyldithio)-propionate, succinmidyl 6-[3-(2-pyridyldithio)propionamido]hexanoate, sulfosuccinmidyl 6-[3-(2-pyridyldithio)propionamido]hexanoate, 1-ethyl-3-(3-dimethylaminopropyl)carbodiimide hydrochloride, N,N′-dicyclohexylcarbodiimide, 1,5-difluoro-2,4-dinitrobenzene, N-5-azido-2-nitrobenzoyloxysuccinimide, N-hydroxysuccinimidyl-4-azidobenzoate, N-hydroxysulfosuccinimidyl-4-azidobenzoate, N-hydroxysuccinimidyl-4-azidosalicylic acid, N-hydroxysulfosuccinimidyl-4-azidosalicylic acid, sulfosuccinimidyl-(4-azidosalicylamido)-hexanoate, p-nitrophenyl-2-diazo-3,3,3-trifluoropropionate, 2-diazo-3,3,3-trifluoropropionylchloride, N-succinimidyl-(4-azidophenyl) 1,3′-dithiopropionate, sulfosuccinimidyl-(4-azidophenyldithio)propionate, sulfosuccinimidyl 2-(7-azido-4-methylcoumarin-3-acetamide) ethyl-1,3′-dithiopropionate, sulfosuccinimidyl 7-azido-4-methylcoumarin-3-acetate, sulfosuccinimidyl 2(m-azido-o-nitrobenzamido)-ethyl-1,3′-dithiopropionate, N-succinimidyl-6-(4′-azido-2′-nitrophenylamino)hexanoate, sulfosuccinimidyl-6-(4′-azido-2′-nitrophenylamino)hexanoate, sulfosuccinimidyl 2-(p-azidosalicylamido)ethyl-1,3′-dithiopropionate, and sulfosuccinimidyl 4-(p-azidophenyl)butyrate and mixtures thereof.

31. The method of claim 30, wherein the capturable moiety of the anchor primer of the double stranded DNA molecule is affixed to a substrate comprising a coating of streptavidin.

32. The method of claim 30, wherein the capturable moiety of the anchor primer of the double stranded DNA molecule is affixed to a substrate comprising a coating of N-oxysuccinimide ester.

33. A method for simultaneous sequence-specific identification of multiple mRNA molecules in a RNA population comprising the steps of:
preparing a population of capturable double-stranded cDNA molecules from a population of mRNA molecules having a 3′ poly (A) terminus by using a mixture of anchor primers, each anchor primer having a 5′ terminus and a 3′ terminus and including: (i) phasing residues located at the 3′ terminus of each of the anchor primers consisting of -V-N-N, wherein V is a deoxyribonucleotide selected from the group consisting of A, C, and G; and N is a deoxyribonucleotide selected from the group consisting of A, C, G, and T, the mixture including anchor primers containing all possibilities for V and N; (ii) a tract of 8 to 40 T residues located towards the 5′-terminus relative to the phasing residues; (iii) a first stuffer segment consisting of 4 to 40 nucleotides; (iv) a segment complementary to a 3′ PCR primer consisting of about 12 to about 20 nucleotide residues located towards the 5′-terminus relative to the tract of T residues; (v) a second stuffer segment consisting of 4 to 40 nucleotides; (vi) at least one segment comprising a sequence recognized by a restriction endonuclease that recognizes at least six bases, the segment being located towards the 5′-terminus of the anchor primer relative to the 3′ PCR primer segment and (vii) a capturable moiety affixed to the anchor primer;
digesting the population of capturable double-stranded cDNA molecules with a restriction endonuclease that recognizes at least a four nucleotide sequence not found within the sequence of the anchor primer, thereby producing a population of capturable double stranded cDNA fragments, each capturable double stranded cDNA fragment having an anchor end that corresponds to the poly(A) segment of the original mRNA molecule and including at least a portion of a sequence corresponding to that of the anchor primer, and a free end opposite to the anchor end;
capturing the capturable moiety, thereby affixing the capturable double-stranded cDNA fragments to a substrate to form affixed double stranded cDNA fragments;
ligating a double stranded adapter polynucleotide to the free end of each affixed double stranded cDNA fragment to form a population of adapted cDNA molecules, the double stranded adapter polynucleotide including a segment corresponding to the sequence of a bacteriophage RNA polymerase promoter and a segment complementary to a 5′ PCR primer;
generating a first set of sequence-specific PCR products by dividing the population of adapted cDNA molecules into a first series of subpools as templates for a first polymerase chain reaction with a 3′ PCR-primer about 15 to 30 nucleotides in length that is complementary to at least a portion of the anchor primer sequence and a first 5′ PCR-primer about 15 to about 30 nucleotides in length and that is complementary to a portion of the adapter polynucleotide, with the complementarity extending one nucleotide beyond the portion of the adapter polynucleotide into the specific sequence corresponding to the free end of the capturable cDNA and including a 3′-terminus consisting of -N_{X}, wherein X is an integer from 1 to 5, and N is selected from group consisting of the four deoxyribonucleotides A, C, G, and T, and wherein a different one of the first 5′ PCR primers is used in each of 4^{X} different subpools;
generating a detectable second set of sequence-specific PCR products by further dividing the first set of sequence-specific PCR products in each of the first series of subpools into a second series of subpools and using the first set of sequence-specific PCR products as templates for a second polymerase chain reaction with a 3′ PCR primer of 15 to 30 nucleotides in length comprising a detectable moiety and a second 5′ PCR primer defined as having a 3′-terminus consisting of -N_{X}-N_{X+Y,} wherein N_{X} is the same as the NX used in the first polymerase chain reaction for that subpool, wherein Y is an integer from 1 to 5, (X+Y) is an integer from 2 to 6, N is selected from group consisting of the four deoxyribonucleotides A, C, G, and T, wherein the second 5′ PCR primer is about 15 to about 30 nucleotides in length and wherein the second 5′ PCR-primer is complementary to a portion of the adapter polynucleotide with the complementarity extending X+Y nucleotides beyond the portion of the adapter polynucleotide into the specific sequence corresponding to the free end of the capturable cDNA, wherein a different one of the second 5′ PCR primers is used in the different 4^{x+Y} subpools of the second series of subpools;
resolving the second set of sequence-specific PCR products to generate a simultaneous display of sequence-specific PCR products representing the 3′-ends of mRNA molecules present in the mRNA population; and
characterizing each sequence-specific PCR product by a partial sequence and a length, thereby providing simultaneous sequence-specific identification of multiple mRNA molecules in a RNA population without making a cDNA library.

34. The method of claim 33, wherein the step of preparing a population of double-stranded cDNA molecules comprises the steps of synthesizing a first cDNA strand and synthesizing a second cDNA strand.

35. The method of claim 33, further comprising the steps of
transcribing to produce synthetic RNA molecules by incubating the capturable cDNA with a bacteriophage RNA polymerase capable of initiating transcription from the sequence corresponding to the sequence of a bacteriophage RNA polymerase promoter; and
generating first-strand cDNA by transcribing the cRNA using a reverse transcriptase and a RT primer being 15 to 30 nucleotides in length and comprising a segment capable of hybridizing to a portion of the anchor primer sequence.

36. The method of claim 33, wherein X=1 and Y=3.

37. The method of claim 33 wherein the anchor primer is chosen from the group consisting of primers having the sequence 5′-A-T-G-A-A-T-T-C-T-C-T-A-G-A-G-A-T-T-G-C-T-A-C-C-T-C-A-G-T-C-T-G-A-G-C-T-C-C-A-C-C-G-C-G-G-T-A-G-T-A-C-T-C-A-C-T-G-C-T-T-T-T-T-T-T-T-T-T-T-T-T-T-T-T-T-T-V-N-N-3′ (SEQ ID NO: 1), 5′-A-T-G-A-A-T-T-C-T-C-T-A-G-A-G-T-C-T-G-A-G-C -T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 2) and 5′-G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 3), wherein the 5′ terminal base (base 1) is a biotinylated adenylate residue, each V can represent A, C or G, and each N can represent A, C, G, or T.

38. The method of claim 33 wherein the bacteriophage RNA polymerase promoter is selected from the group consisting of T3 promoter, T7 promoter and SP6 promoter.

39. The method of claim 33 wherein one strand of the double stranded adapter polynucleotide comprises the sequence 5′-A-T-G-A-A-T-T-C-G-G-T-A-C-C-A-A-T-T-A-A-C-C-C-T-C-A-C-T-A-A-A-G-G-G-A-C-A-G-C-T-T-A-T-C-A-T-C-G-C-T-C-G-A-G-C-T-C-G-A-C-G-G-T-A-T-3′ (SEQ ID NO:7).

40. The method of claim 33 wherein one strand of the double stranded adapter polynucleotide comprises the sequence 5′-C-G-A-T-A-C-C-G-T-C-G-A-G-C-T-C-G-A-G-C-G-A-T-G-A-T-A-A-G-C-T-G-T-C-C-C-T-T-T-A-G-T-G-A-G-G-G-T-T-A-A-T-T-G-G-T-A-C-C-G-A-A-T-T-C-A-T-3′ (SEQ ID NO:8).

41. The method of claim 33 wherein one strand of the double stranded adapter polynucleotide comprises the sequence 5′-Phospho-C-G-A-T-A-C-C-G-T-C-G-A-C-C-T-C-G-A-G-G-T-C-C-C-T-T-T-A-G-T-G-A-G-G-G-T-T-A-A-T-T-G-G-T-A-C-C-G-A-A-T-T-3′ (SEQ ID NO:9).

42. The method of claim 33 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-G-G-T-C-G-A-C-G-G-T-A-T-3′ (SEQ ID NO: 10).

43. The method of claim 33 wherein one strand of the double stranded adapter polynucleotide comprises the sequence 5′-Phospho-G-A-T-C-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO: 11).

44. The method of claim 33 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-3′ (SEQ ID NO:12).

45. The method of claim 33 wherein one strand of the double stranded adapter polynucleotide comprises the sequence 5′-Phospho-C-T-C-A-C-C-A-C-A-G-A-G-C-T-T-C-G-A-G-G-T-C-C-C-T-T-T-A-G-T-G-A-G-G-G-T-T-A-A-T-T-G-G-T-A-C-C-G-A-A-T-T-3′ (SEQ ID NO: 13).

46. The method of claim 33 wherein one strand of the double stranded adapter polynucleotide comprises the sequence 5′-A-A-T-T-C-G-G-T-A-C-C-A-A-T-T-A-A-C-C-C-T-C-A-C-T-A-A-A-G-G-G-A-C-C-T-C-G-A-A-G-C-T-C-T-G-T-G-G-T-G-A-G-C-A-T-G-3′ (SEQ ID NO:14).

47. The method of claim 33 wherein the 3′ PCR primer comprises a sequence chosen from the group consisting of 5′-G-A-G-C-T-C-C-A-C-C-G-C-G-G-T-3′ (SEQ ID NO:5)) and 5′-G-A-G-C-T-C-G-T-T-T-T-C-C-C-A-G-3′ (SEQ ID NO:6).

48. The method of claim 33 wherein the RT primer comprises a sequence chosen from the group consisting of 5′-C-A-G-T-C-T-G-A-G-C-T-C-C-A-C-C-G-C-G-G-T-3′ (SEQ ID NO:15) and 5′-G-A-G-C-T-C-C-A-C-C-G-C-G-G-T-3′ (SEQ ID NO:5).

49. The method of claim 33 wherein the first 5′ PCR primer comprises a sequence chosen from the group consisting of 5′-C-T-C-G-A-G-C-T-C-G-A-C-G-G-T-A-T-C-G-G-N-3′ (SEQ ID NO:16), 5′-C-C-T-C-G-A-G-G-T-C-G-A-C-G-G-T-A-T-C-G-G-N-3′ (SEQ ID NO:17), 5′ A-G-C-T-C-T-G-T-G-G-T-G-A-G-G-A-T-C-N-3′ (SEQ ID NO:19), 5′-A-G-C-T-C-T-G-T-G-G-T-G-A-G-C-A-T-G-N-3′ (SEQ ID NO:21) and 5′-C-C-T-C-G-A-G-G-T-C-G-A-C-G-G-T-A-T-C-G-A-N -3′ (SEQ ID NO:23).

50. The method of claim 33 wherein the second 5′ PCR primer comprises a sequence chosen from the group consisting of 5′-C-G-A-C-G-G-T-A-T-C-G-G-N-N-N-N-3′ (SEQ ID
NO: 18), 5′-C-T-C-T-G-T-G-G-T-G-A-G-G-A-T-C-N-N-N-N-3′ (SEQ ID NO:20), 5′-C-T-C-T-G-T-G-G-T-G-A-G-C-A-T-G-N-N-N-N-3′ (SEQ ID NO:22), and 5′-T-C-G-A-G-G-T-C-G-A-C-G-G-T-A-T-C-G-A-N-N-N-N-3′ (SEQ ID NO:24).

51. The method of claim 33 wherein the restriction endonuclease recognizing at least a four nucleotide sequence not found within the sequence of the anchor primer is selected from the group consisting of AciI, AluI, BfaI, BstUI, Csp6I, DpnI, DpnII, HaeIII, HhaI, HinP1I, HpaII, MaeII, MboI, MnlI, MseI, MspI, NlaIII, RsaI, Sau3AI, TaiI, TaqI, and Tsp509I.

52. The method of claim 33 wherein the restriction endonuclease recognizing a four-nucleotide sequence is Msp I

53. The method of claim 33 wherein the restriction endonuclease recognizing at least a four nucleotide sequence not found within the sequence of the anchor primer is selected from the group consisting of BglII, BmgI, BsaAI, BsaHI, BsaWI, BsbI, BsePI, BseSI, BsiI, BsiEI, BsiHKAI, BsiWI, BsmI, Bsp1286I, Bsp1407I, BspEI, BspGI, BspHI, BspLU11I, BspEI, BspGI, BspHI, BspLU11I, BspMII, BsrBI, BsrFI, BsrGI, BssHII, BssHII, BssSI, BstBI, BstYI, BstZ17I, BtgI, BtrI, CfrI, Cfr10I, ClaI, DraI, DrdII, DsaI, EaeI, EagI, Ecl136II, Eco47III, EcoNI, EspI, Esp3I, EspI, GdiII, HaeI, HaeII, HgiAI, HgiEII, HgiJII, Hin4I, HincII, HindII, HindIII, KasI, KpnI, Ksp632I, LpnI, MfeI, MmeI, MscI, MslI, MspA1I, MstI, NaeI, NarI, NcoI, NdeI, NheI, Nli3877I, NotI, NruI, NspBII, OliI, PciI, PflMI, PmeI, Ppu10MI, PpuMI, PspOMI, PsrI, PssI, PvuII, RleAI, RsrII, SapI, SauI, SbfI, SciI, SduI, SfcI, SfoI, SgfI, SgrAI, SmaI, SmlI, SnaI, SnaBI, SrfI, Sse232I, Sse8387I, Sse8647I, StyI, Tth111I, Tth111II, UbaKI, VspI, XbaI, XcmI, XhoI, XmaI.

54. The method of claim 33 wherein the restriction endonuclease recognizing at least a four nucleotide sequence not found within the sequence of the anchor prime is selected from the group consisting of AscI, BaeI, FseI, NotI, PacI, PmeI, PpuMI, RsrII, SapI, SexAI, SfiI, SgfI, SgrAI, SrfI, Sse8387I and SwaI.

55. The method of claim 33 wherein the restriction endonuclease that recognizes at least six bases is selected from the group consisting of AscI, BaeI, FseI, NotI, PacI, PmeI, PpuMI, RsrII, SaI, SexAI, SfiI, SgfI, SgrAI, SrfI, Sse8387I and SwaI.

56. The method of claim 33 wherein the restriction endonuclease that recognizes at least six bases is selected from the group consisting of EcoRl and XbaI.

57. The method of claim 33 wherein the capturable moiety of the anchor primer of the double stranded DNA molecule is biotin.

58. The method of claim 33 wherein the detectable moiety of the anchor primer of the second set of sequence-specific PCR products is a fluorescent moiety and is detected by monitoring laser-induced fluorescent emission.

59. The method of claim 33 wherein the restriction endonuclease that recognizes at least a four nucleotide sequence not found within the sequence of the anchor primer is MspI and the first 5′ PCR primer comprises the sequence 5′-C-T-C-G-A-G-C-T-C-G-A-C-G-G-T-A-T-C-G-G-N-3′ (SEQ ID NO:16).

60. The method of claim 33 wherein the first 5′ PCR primer comprises the sequence 5′-C-C-T-C-G-A-G-G-T-C-G-A-C-G-G-T-A-T-C-G-G-N-3′ (SEQ ID NO:17) and the second 5′ PCR primer comprises the sequence 5′-C-G-A-C-G-G-T-A-T-C-G-G-N-N-N-N-3′ (SEQ ID NO:18).

61. The method of claim 33 wherein the restriction endonuclease that recognizes at least a four nucleotide sequence not found within the sequence of the anchor primer is Sau3AI, the first 5′ PCR primer comprises the sequence 5′ A-G-C-T-C-T-G-T-G-G-T-G-A-G-G-A-T-C-N-3′ (SEQ ID NO: 19), and the second 5′ PCR primer comprises the sequence 5′-C-T-C-T-G-T-G-G-T-G-A-G-G-A-T-C-N-N-N-N-3′ (SEQ ID NO:20).

62. The method of claim 33 wherein the restriction endonuclease that recognizes at least a four nucleotide sequence not found within the sequence of the anchor primer is NlaIII, the first 5′ PCR primer comprises the sequence 5′-A-G-C-T-C-T-G-T-G-G-T-G-A-G-C-A-T-G-N-3′ (SEQ ID NO:21), and the second 5′ PCR primer comprises the sequence 5′-C-T-C-T-G-T-G-G-T-G-A-G-C-A-T-G-N-N-N-N-3′ (SEQ ID NO:22).

63. The method of claim 33 wherein the restriction endonuclease that recognizes at least a four nucleotide sequence not found within the sequence of the anchor primer is TaqI, the first 5′ PCR primer comprises the sequence 5′-C-C-T-C-G-A-G-G-T-C-G-A-C-G-G-T-A-T-C-G-A-N -3′ (SEQ ID NO:23), the second 5′ PCR primer comprises the sequence 5′-T-C-G-A-G-G-T-C-G-A-C-G-G-T-A-T-C-G-A-N-N-N-N-3′ (SEQ ID NO:24).

64. A set of anchor primers having the sequence 5′-A-T-G-A-A-T-T-C-T-C-T-A-G-A-G-A-T-T-G-C-T-A-C-C-T-C-A-G-T-C-T-G-A-G-C-T-C-C-A-C-C-G-C-G-G-T-A-G-T-A-C-T-C-A-C-T-G-C-T-T-T-T-T-T-T-T-T-T-T-T-T-T-T-T-T-T-V-N-N-3′ (SEQ ID NO: 1) wherein the 5′ terminal base (base 1) is a biotinylated adenylate residue, V can represent A, C or G, and each N can represent A, C, G, or T.

65. A set of anchor primers having the sequence 5′-A-T-G-A-A-T-T-C-T-C-T-A-G-A-G-T-C-T-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 2) wherein the 5′ terminal base (base 1) is a biotinylated adenylate residue, V can represent A, C or G, and each N can represent A, C, G, or T.

66. A set of anchor primers having the sequence 5′-G-A-A-T-T-C-A-A-C-T-G-G-A-A-G-C-G-G-C-C-G-C-A-G-G-A-A-G-A-G-C-T-C-C-A-C-C-G-C-G-G-T-A-G-T-A-C-T-C-A-C-T-G-C-A-G-T-T-T-T-T-T-T-T-T-T-T-T-T-T-T-T-T-T-V-N-N-3′ (SEQ ID NO: 3) wherein the 5′ terminal base (base 1) is a biotinylated guanosine residue, V can represent A, C or G, and each N can represent A, C, G, or T.

67. A system for storing and displaying characteristics of polynucleotide fragments comprising, in combination:
a graphical user interface for visually displaying characteristics of polynucleotide fragments and
at least one database for storing characteristics of polynucleotide fragments stored on a computer readable medium.

68. The system of claim 67 wherein at least one database comprises data produced by the quantitation of the fragment length and relative abundance of sequence-specific PCR products.

69. The system of claim 68 wherein the sequence-specific PCR products are differentially expressed sequence-specific PCR products.

70. The system of claim 67 wherein at least one database comprises data including sequence relationships, gene mapping and cellular distributions.

71. The system of claim 67 wherein at least one database comprises data including corresponding nucleotide sequences determined from a general database of nucleotide sequences, the corresponding nucleotide sequences being delimited by the 3′-most recognition site for the restriction endonuclease that recognizes at least a four nucleotide sequence not found within the sequence of the anchor primer and the beginning of the poly(A) tail.

72. The system of claim 71 wherein at least one database comprises data including an expected length of a sequence-specific PCR product.

73. The system of claim 72 wherein the expected length of a sequence-specific PCR product is determined from the sum of the lengths of the corresponding nucleotide sequence determined from the general database, the length of the 5′ PCR sequence hybridizable to the adapter sequence, and the length of the remaining anchor primer sequence, including the length of the 3′ PCR primer segment.

74. The system of claim 73 further comprising a set of executable instructions stored on a computer readable medium suitable for comparing characteristics of the sequence-specific PCR products to characteristics of the corresponding nucleotide sequence determined from the general database.


SEQUENCE LISTING (32 sequences)

SEQ ID NO: 1 - DNA, 79 bp, Artificial Sequence
  Description of Artificial Sequence synthetic primer (cDNA anchor primer) wherein base 1 is a biotinylated adenosine residue
atgaattctctagagattgctacctcagtctgagctccaccgcggtagtactcactgcttttttttttttttttttvnn

SEQ ID NO: 2 - DNA, 48 bp, Artificial Sequence
  Description of Artificial Sequence  synthetic primer (cDNA anchor primer)
gaattcaactggaagcggccgcaggaattttttttttttttttttvnn

SEQ ID NO: 3 - DNA, 15 bp, Artificial Sequence
  Description of Artificial Sequence 3′ RT primer
gagctccaccgcggt

SEQ ID NO: 4 - DNA, 16 bp, Artificial Sequence
  Description of Artificial Sequence 3′ PCR primer
gagctcgttttcccag

SEQ ID NO: 5 - DNA, 65 bp, Artificial Sequence
  Description of Artificial Sequence one strand of double stranded adapter
atgaattcggtaccaattaaccctcactaaagggacagcttatcatcgctcgagctcgacggtat

SEQ ID NO: 6 - DNA, 67 bp, Artificial Sequence
  Description of Artificial Sequence other strand of double stranded adapter
cgataccgtcgagctcgagcgatgataagctgtccctttagtgagggttaattggtaccgaattcat

SEQ ID NO: 7 - DNA, 52 bp, Artificial Sequence
  Description of Artificial SequenceO1 (antisense strand); double stranded adapter wherein base 1 is a phosphorylated cytosine residue
cgataccgtcgacctcgaggtccctttagtgagggttaattggtaccgaatt

SEQ ID NO: 8 - DNA, 50 bp, Artificial Sequence
  Description of Artificial Sequence O2 (sense strand); double stranded adapter
aattcggtaccaattaaccctcactaaagggacctcgaggtcgacggtat

SEQ ID NO: 9 - DNA, 56 bp, Artificial Sequence
  Description of Artificial Sequence one strand of double stranded adapter wherein base 1 is a phosphorylated guanosine residue
gatcctcaccacagagcttcgaggtccctttagtgagggttaattggtaccgaatt

SEQ ID NO: 10 - DNA, 52 bp, Artificial Sequence
  Description of Artificial Sequence one strand of double stranded adapter
aattcggtaccaattaaccctcactaaagggacctcgaagctctgtggtgag

SEQ ID NO: 11 - DNA, 52 bp, Artificial Sequence
  Description of Artificial Sequence one strand of a double stranded adapter wherein base 1 is a phosphorylated cytosine residue
ctcaccacagagcttcgaggtccctttagtgagggttaattggtaccgaatt

SEQ ID NO: 12 - DNA, 56 bp, Artificial Sequence
  Description of Artificial Sequence one strand of double stranded adapter
aattcggtaccaattaaccctcactaaagggacctcgaagctctgtggtgagcatg

SEQ ID NO: 13 - DNA, 20 bp, Artificial Sequence
  Description of Artificial Sequence Reverse transcriptase MN0 primer
cagtctgagtccaccgcggt

SEQ ID NO: 14 - DNA, 21 bp, Artificial Sequence
  Description of Artificial Sequence synthetic primer (5′ PCR N1 primer)
ctcgagctcgacggtatcggn

SEQ ID NO: 15 - DNA, 16 bp, Artificial Sequence
  Description of Artificial Sequence synthetic primer (5′ PCR N4 primer)
cgacggtatcggnnnn

SEQ ID NO: 16 - DNA, 19 bp, Artificial Sequence
  Description of Artificial Sequence synthetic primer (5′ PCR N1 primer)
agctctgtggtgaggatcn

SEQ ID NO: 17 - DNA, 16 bp, Artificial Sequence
  Description of Artificial Sequence synthetic primer (5′ PCR N4 primer)
gtggtgaggatcnnnn

SEQ ID NO: 18 - DNA, 19 bp, Artificial Sequence
  Description of Artificial Sequence synthetic primer (5′ PCR N1 primer)
agctctgtggtgagcatgn

SEQ ID NO: 19 - DNA, 16 bp, Artificial Sequence
  Description of Artificial Sequence synthetic primer (5′ PCR N4 primer)
gtggtgagcatgnnnn

SEQ ID NO: 20 - DNA, 22 bp, Artificial Sequence
  Description of Artificial Sequence synthetic primer (5′ PCR N1 primer)
cctcgaggtcgacggtatcgan

SEQ ID NO: 21 - DNA, 16 bp, Artificial Sequence
  Description of Artificial Sequence synthetic primer (5′ PCR N4 primer)
cgacggtatcgannnn

SEQ ID NO: 22 - DNA, 30 bp, Artificial Sequence
  Description of Artificial Sequence synthetic primer (NF-kB extended primer)
gatcgaatccggcccgcctgaatcattctc

SEQ ID NO: 23 - DNA, 12 bp, Artificial Sequence
  Description of Artificial Sequence first stuffer segment of anchor primer
agtactcactgc

SEQ ID NO: 24 - DNA, 17 bp, Artificial Sequence
  Description of Artificial Sequence second stuffer segment of anchor primer
gattgctacctcagtct

SEQ ID NO: 25 - DNA, 16 bp, Artificial Sequence
  Description of Artificial Sequence synthetic primer (5′ PCR N4 primer)
gctcgacggtatcggn

SEQ ID NO: 26 - DNA, 16 bp, Artificial Sequence
  Description of Artificial Sequence synthetic primer (5′ PCR N2 primer)
ctcgacggtatcggnn

SEQ ID NO: 27 - DNA, 16 bp, Artificial Sequence
  Description of Artificial Sequence synthetic primer (5′ PCR N3 primer)
tcgacggtatcggnnn

SEQ ID NO: 28 - DNA, 16 bp, Artificial Sequence
  Description of Artificial Sequence synthetic primer (5′ PCR N5 primer)
gacggtatcggnnnnn

SEQ ID NO: 29 - DNA, 16 bp, Artificial Sequence
  Description of Artificial Sequence synthetic primer (5′ PCR N6 primer)
acggtatcggnnnnnn

SEQ ID NO: 30 - DNA, 16 bp, Artificial Sequence
  Description of Artificial Sequence synthetic primer (5′ PCR N4 primer)
ggtcgacggtatcggn

SEQ ID NO: 31 - DNA, 16 bp, Artificial Sequence
  Description of Artificial Sequence synthetic primer (5′ RT primer)
aggtcgacggtatcgg

SEQ ID NO: 32 - DNA, 21 bp, Artificial Sequence
  Description of Artificial Sequence synthetic primer (5′ RT primer)
gagctcgacggtatcggnnnn